(19) United States
(12) Patent Application Publication
(10) Pub. No.: US 2026/0338787 A1
(43) Pub. Date: Oct. 29, 2026

(54) BIOMARKER COMPOSITION FOR DIAGNOSING AGING CONTAINING MITORIBOSOME PROTEIN, AND USE THEREOF

(71) Applicant: Ajou University Industry-Academic Cooperation Foundation, Gyeonggi-do (KR)

(72) Inventors: Subin LIM, Gyeonggi-do (KR); Gyesoon YOON, Gyeonggi-do (KR)

(73) Assignee: Ajou University Industry-Academic Cooperation Foundation, Gyeonggi-do (KR)

(21) Appl. No.: 19/109,637

(22) PCT Filed: Jun. 16, 2023
(86) PCT No.: PCT/KR2023/008350

§ 371 Date: Mar. 7, 2025

(30) Foreign Application Priority Data
Sep. 8, 2022 (KR) 10-2022-0114429

(51) Int. Cl. G01N 33/68

(52) U.S. Cl. CPC G01N 33/68; G01N 2800/7042

(57) ABSTRACT

The present invention relates to: a biomarker composition for diagnosing senescence which can be utilized as a biomarker for diagnosing senescence by identifying the expression level of a mitoribosomal protein; and a use thereof.

The mitoribosomal protein of the present invention shows a decreased expression level in the early stages of senescence, and thus can be effectively utilized in a biomarker composition for diagnosing senescence, a kit for diagnosing senescence including same, or a method for providing information for diagnosing senescence.

### TECHNICAL FIELD

This application claims priority to and the benefit of Korean Patent Application No. 10-2022-0114429, filed on Sep. 8, 2022, the disclosure of which is incorporated herein by reference in its entirety.

The present invention relates to a biomarker composition for diagnosing senescence that can be utilized as a biomarker for diagnosing senescence by determining the expression levels of mitoribosomal proteins, and uses thereof.

The present invention was completed with the support of the Ministry of Science and ICT and the Ministry of Education of the Republic of Korea under Project No. 2020M3A9D8038009 (1711158350) and Project No. 2020R1A6A1A03043539 (1345347233).

### BACKGROUND ART

Mitochondrial oxidative phosphorylation (OXPHOS)-mediated bioenergetic deregulation has long been implicated in cellular and organismal senescence processes, thereby supporting the mitochondrial free radical senescence theory. Mitochondria have their own DNA and independent genetic information transfer systems such as replication, transcription and translation machinery, and these are collectively referred to as the mitochondrial central dogma (MCD) system. The core effector proteins encoded by nuclear DNA (ncDNA) involved in MCD activity are fundamentally different from the central dogma system of ncDNA origin. Mammalian mitochondrial DNA (mtDNA), which is a circular double-stranded DNA length of approximately 16 kb, is highly vulnerable to oxidative stress due to its chromatin-like unpacked nuclear-type structure and proximity to reactive oxygen species (ROS)-generating OXPHOS sites. The accumulation of somatic mutations and/or deletions in mtDNA is considered as an important contributor to senescence and senescence processes, which was further demonstrated by studies in DNA polymerase-deficient mice. Nevertheless, it is still unclear whether mtDNA damage is the sole cause of senescence-related mitochondrial dysfunction and how MCD activity is involved in dysfunction. Although there are only 13 mtDNA-encoded proteins synthesized by MCD activity, these protein products are essential components of the OXPHOS system that governs aerobic ATP production. The final executor of the MCD, the mitochondrial ribosome (mitoribosome), is a specialized large structure that carries out the translation of 13 proteins. Structurally, mitoribosomes are formed by 82 ncDNA-encoded mitoribosomal proteins (MRPs) and 2 mtDNA-encoded ribosomal RNAs (rRNAs), and this suggests that the well-organized expression of 82 MRPS and 2 rRNAs is important in regulating mitochondrial activity.

After a limited number of population doublings, primary human diploid fibroblasts (HDFs) enter a state called replicative senescence (RS), and in this state, growth is irreversibly arrested. RS has been used as a well-established in vitro cellular senescence model to investigate the normal process of organismal senescence. The most extensively studied stimulus leading to RS is telomere attrition due to the “end-replication problem” during DNA replication. This accelerated the senescence process by inhibiting mitochondrial biogenesis through proliferator-activated receptor γ-coactivator (PGC)-1 and/or PGC-1 through p53, mTOR or PARP activation, thereby creating a telomere-mitochondrial axis. On the other hand, telomeric G triplets are particularly sensitive to cleavage during oxidative damage, and it thus suggested that mitochondrial deregulation-induced ROS contribute to telomere attrition. Human telomeres are assembled into a characteristic multiprotein complex called shelterin, which is composed of numerous copies of 6 proteins: telomere-associated factor 1 (TRF1), TRF2, repressor/activator protein 1 (RAP1), protecting telomere protein 1 (POT1), TRF1-interacting nucleoprotein 2 (TIN2) and TIN2- and POT1-interacting protein (TPP1). Shelterin protects telomere sequences by regulating both telomeric DNA structure and telomerase activity. p53-dependent TRF2 degradation and human antigen R (HuR) loss-mediated TIN2 destabilization have been reported to contribute to the induction and maintenance of cellular senescence. These findings suggest that the altered expression or activity of shelterin factors regulates cellular senescence through telomere deprotection-mediated damage.

Senescent cells accumulate in mammalian tissues at varying rates and in an asynchronous manner with senescence. The inventors of the present invention previously reported that during RS, HDFs progressively undergo four stages of senescence with distinct gene expression profiles, and this suggests a gradual and not rapid progression to senescence.

### RELATED ART DOCUMENTS

### Non-Patent Documents

(Non-Patent Document 1) Kim, Y. M.; Byun, H. O.; Jee, B. A.; Cho, H.; Seo, Y. H.; Kim, Y. S.; Park, M. H.; Chung, H. Y.; Woo, H. G.; Yoon, G. Implications of time-series gene expression profiles of replicative senescence. Aging Cell 2013, 12, 622-634.
(Non-Patent Document 2) Jung, H. J.; Byun, H. O.; Jee, B. A.; Min, S.; Jeoun, U. W.; Lee, Y. K.; Seo, Y.; Woo, H. G.; Yoon, G. The Ubiquitin-like with PHD and Ring Finger Domains 1 (UHRF1)/DNA Methyltransferase 1 (DNMT1) Axis Is a Primary Regulator of Cell Senescence. J. Biol. Chem. 2017, 292, 3729-3739.

### DISCLOSURE

### Technical Problem

As a result of our diligent efforts to provide a powerful senescence-specific biomarker, the inventors of the present invention confirmed that mitoribosomal deregulation is a major cause of cellular senescence, and identified mitoribosomal proteins that can be utilized as senescence-related biomarkers because the expression level thereof decreases in the early stages of senescence, thereby completing the present invention

Therefore, an object of the present invention is to provide a biomarker composition for diagnosing senescence that can be utilized as a biomarker for diagnosing senescence by determining the expression levels of mitoribosomal proteins, and uses thereof.

### Technical Solution

The present invention provides a biomarker composition for diagnosing senescence, including an agent for detecting any one or more mitoribosomal proteins selected from the group consisting of MRPL21, MRPL28, MRPS17, MRPL40, MRPL58, MRPS12, MRPS2 and MRPS7, or a gene encoding the mitoribosomal protein.

According to a preferred embodiment of the present invention, the senescence is in early stages of senescence.

According to a preferred embodiment of the present invention, the early stages of senescence are before activation of senescence β-galactosidase (SA-β-gal).

According to a preferred embodiment of the present invention, the senescence is skin senescence.

The present invention provides a kit for diagnosing senescence, including the biomarker composition.

The present invention provides a method for providing information for diagnosing senescence, including:
i) measuring an expression level of any one or more mitoribosomal proteins selected from the group consisting of MRPL21, MRPL28, MRPS17, MRPL40, MRPL58, MRPS12, MRPS2 and MRPS7, or a gene encoding the mitoribosomal protein in a sample isolated from a subject;
ii) comparing the measured expression level with a control sample; and
iii) classifying the measured expression level as senescence if the measured expression level is lower than an expression level of the control sample.

The ‘replicative senescence (RS)’ of the present invention may refer to an irreversible proliferation arrest state that human somatic cells or human diploid fibroblasts eventually reach by dividing in culture.

The ‘oxidative stress-induced senescence (OS) or hydrogen peroxide-induced senescence’ of the present invention may refer to senescence induced by treating human somatic cells or human diploid fibroblasts with hydrogen peroxide (H₂O₂).

The ‘early stages of senescence’ of the present invention may mean a stage before the expression level of SA-β-gal increases, or an early stage (E) when the senescence of human somatic cells or human diploid fibroblasts is classified into four stages: early (E), middle (M), advanced (A) and very advanced (VA) according to [Non-Patent Document 1].

By utilizing diverse mitochondria-related gene expression profiles derived from two different models of cellular senescence in human diploid fibroblasts, the present invention confirmed that the expression of mitoribosomal proteins (MRPs) generally decreased during the early to mid-transition period before the onset of prominent SA-β-gal activity. The suppressed expression patterns of confirmed senescence-associated MRP signatures (SA-MRPs) were validated at single-cell resolution in aged human cells, rat and mouse skin tissues, and aged mouse fibroblasts. TIN2 and POT1-interaction proteins (TPP1, TIN2- and POT1-interaction proteins) were simultaneously suppressed, thereby inducing senescence along with telomeric DNA damage. Finally, the inventors of the present invention showed that SA-MRP deregulation could be a potential upstream regulator of TPP1 inhibition. The present invention has shown that mitoribosomal deregulation may represent an early event initiating mitochondrial dysfunction and may serve as a key driver of cellular senescence and upstream regulator of shelterin-mediated telomere deprotection

Currently, SA-β-gal represents the most widely used biomarker for senescent cells, but it is followed by other senescent phenotypes such as increases in ROS, cell size and cell granularity that occur in the later stages of senescence. Moreover, these early phenotypes are accompanied by early genetic reprogramming, thereby opening new avenues for developing specific genetic markers for early stages of senescence. In the present invention, through comprehensive analysis of transcriptomic data for two senescence models (RS and OS), the inventors of the present invention confirmed a collective MRP deregulation that occurs during the E-M phase transition before the appearance of prominent SA-β-gal activity, thereby completing present invention. 51 SA-MRPs were identified as key features for early entry into HDF senescence. By using publicly available transcriptome analysis of aged human primary fibroblasts, mouse dermal fibroblasts and newly generated validation data derived from aged mouse and rat skin tissue, the inventors of the present invention also discovered that collective MRP deregulation is a common event for cellular senescence and senescence processes, and identified 15 deregulated SA-MRP genes (MRPL21, MRPL22, MRPL27, MRPL28, MRPL40, MRPL46, MRPL48, MRPL58, MRPS12, MRPS17, MRPS18B, MRPS2, MRPS26, MRPS33 and MRPS7) as shared features of MRP deregulation across diverse species and cell types.

Mitoribosomes are macrostructures of dual genetic origin including 2 mtDNA-encoded mitoribosome RNAs and 82 nuclear DNA-encoded MRPs, and they are specialized in the translation of proteins encoded by 13 mtDNAs that are key to OXPHOS, which governs mitochondrial energy. The inventors of the present invention also demonstrated that the individual knockdown of several MRPs (MRPS9, MRPS15 and MRPS31) in primary young HDFs caused senescence with a decrease in mitochondrial translation activity and an increase in mitochondrial ROS. These findings indicate that MRP deregulation-mediated mitochondrial dysfunction may represent an early event that promotes the entry of cells into senescence.

Since cellular senescence, which is established by extensive in vitro cell culture, was first described by Hayflick in the 1960s, the RS of HDFs with finite cell replicative lifespans has been widely used in cellular modeling of human senescence. The main underlying mechanism of RS has been described as telomere attrition or telomere shortening, which is a result of the “end-replication problem” during repeated cell divisions. Despite the inevitable telomere attrition induced by the “end-replication problem” in mitotic cells, the repetitive telomeric DNA sequences have a protection mechanism involving the shelterin complex, which is composed of 6 proteins (TPP1, POT1, RAP1, TIN2, TRF1 and TRF2). Multiple shelterin complexes bind to telomeric DNA sequences, thereby protecting the structure through T-loop formation and recruiting telomerase enzymes when telomere elongation is required. TPP1 is known to play an essential function in constituting a shelterin complex by promoting the interaction between TIN2, TRF1 and TRF2 and mediating POT1 recruitment to telomeres. As a result, TPP1 recruits and regulates telomerase activity for telomere protection. Conditional knockout experiments demonstrated a role for TPP1 in protecting telomeres through POT1 recruitment. In the present invention, the inventors of the present invention confirmed that telomere attrition occurred during the E to M transition of RS progression, which was minimally and progressively amplified in the M phase. The mRNA and protein levels of TPP1 were clearly repressed at the E to M transition and remained consistent thereafter, indicating that the M phase shelterin complex had a limited capacity for telomerase recruitment even though telomerase levels were unchanged. The inventors of the present invention also demonstrated that TPP1 inhibition induces senescence accompanied by telomeric DNA damage without significant changes in telomere length and MRP deregulation functions as an upstream repressor of TPP1 expression.

The present invention suggested that in the RS model, two events of telomere attrition related to the “end-replication problem” and telomeric DNA damage caused by TPP1 inhibition can occur in the M stage with a population doubling (PD) number of 60 or more. (FIGS. 1a and 4a), and TPP1-mediated telomeric DNA damage alone was able to induce senescence without telomere attrition. The inventors of the present invention hypothesized that the absence of telomere attrition in TPP1 inhibition-induced senescence may be due to a short experimental period (4 days) to create an “end-replication problem.”

In vitro data from primary HDFs show that MRP deregulation can induce premature senescence, but this cellular feature was not sufficient to explain how even unicellular types in organismal tissues could be involved in tissue and organismal senescence progression due to cellular heterogeneity in the senescence stages. In order to solve this problem, the inventors of the present invention analyzed publicly available scRNA-seq data sets derived from aged mouse dermal fibroblasts and observed the presence of 5 distinct cell subpopulations (C1, C2, C3, C4 and C5) suggesting heterogeneous age-defining characteristics of cell subpopulations, and the diverse composition of these subpopulations in mice of different compositions. Importantly, the inventors of the present invention discovered that the expression levels of MRP and shelterin genes gradually decreased from C1 to C5. Herein, C1 and C2 cells were mostly derived from newborn mice (1.5/2.5 days old), whereas similar numbers of C3 and C4 cells were obtained from young (2 months old) and old (18 months old) mice. The inventors of the present invention speculated that MRP and shelterin genes, including Acd, are repressed between newborns and young ages. However, since the analyzed data sets were derived only from mice having three differently ages (newborn (P1.5-2.5 days), young (2 months) and old (18 months)), the exact point of senescence at which MRP and shelterin genes are repressed could not be determined. Collectively, the inventors of the present invention report that MRP deregulation inducing TPP1 inhibition is an early entry into human cell senescence, and provide 15 SA-MRPs and TPP1 as novel markers of early senescence that cannot be characterized by SA-β-gal activity. Finally, the inventors of the present invention revealed the functional importance of TPP1-mediated telomere deprotection in senescence induction.

Therefore, the present invention may provide a biomarker composition for diagnosing senescence, including an agent for detecting any one or more mitoribosomal proteins selected from the group consisting of MRPL21, MRPL28, MRPS17, MRPL40, MRPL58, MRPS12, MRPS2 and MRPS7, or a gene encoding the mitoribosomal protein.

The ‘agent for detecting a protein’ refers to a substance that can be used to detect the mitoribosomal protein. The agent capable of specifically detecting the protein may be antibodies, antibody fragments, antibody mimetics, aptamers, avidity multimers or peptidomimerics. A method of producing an antibody using a mitoribosomal protein may be easily prepared by a person skilled in the art using techniques known in the art.

The ‘agent for detecting a gene’ refers to a substance that can be used to specifically identify or detect genes encoding the mitoribosomal proteins, such as any one or more selected from the group consisting of MRPL21, MRPL28, MRPS17, MRPL40, MRPL58, MRPS12, MRPS2 and MRPS7. The agent capable of specifically detecting the gene may be primers, probes or antisense nucleic acids.

According to a preferred embodiment of the present invention, the senescence may be in early stages of senescence.

According to a preferred embodiment of the present invention, the early stages of senescence may be before the activation of senescence β-galactosidase (SA-β-gal).

According to a preferred embodiment of the present invention, the senescence may be skin senescence.

In addition, the present invention may provide a kit for diagnosing senescence, including the biomarker composition.

According to a preferred embodiment of the present invention, the senescence may be in early stages of senescence.

According to a preferred embodiment of the present invention, the early stages of senescence may be before the activation of senescence β-galactosidase (SA-β-gal).

According to a preferred embodiment of the present invention, the senescence may be skin senescence.

The kit of the present invention may be composed of one or more different component compositions, solutions or devices suitable for commonly used detection methods. For example, a kit for measuring protein expression levels may include a substrate, an appropriate buffer solution, a secondary antibody labeled with a chromogenic enzyme or a fluorescent substance, a chromogenic substrate and the like for the immunological detection of an antibody.

The kit of the present invention may include a sample extraction means for obtaining a sample from an evaluation subject. The sample extraction means may include a needle or syringe. The kit may include a sample collection container to contain the extracted sample, which may be a liquid, a gas or a semi-solid. The kit may further include instructions for use. The sample may be any body sample in which mitoribosomal proteins may be present or secreted. The measurement of mitoribosomal proteins in body samples may be performed on whole samples or processed samples.

In addition, the present invention may provide a method for providing information for diagnosing senescence, including:
i) measuring an expression level of any one or more mitoribosomal proteins selected from the group consisting of MRPL21, MRPL28, MRPS17, MRPL40, MRPL58, MRPS12, MRPS2 and MRPS7, or a gene encoding the mitoribosomal protein in a sample isolated from a subject;
ii) comparing the measured expression level with a control sample; and
iii) classifying the measured expression level as senescence if the measured expression level is lower than an expression level of the control sample.

The sample may be tissue, cells, whole blood, serum, plasma, saliva, urine, stool, sweat or cerebrospinal fluid. More preferably, the sample may be tissue or cells.

The control group may refer to a sample in which senescence has not progressed.

According to a preferred embodiment of the present invention, the senescence may be in early stages of senescence.

According to a preferred embodiment of the present invention, the early stages of senescence may be before the activation of senescence β-galactosidase (SA-β-gal).

According to a preferred embodiment of the present invention, the senescence may be skin senescence.

### Advantageous Effects

Since the expression level of the mitoribosomal protein of the present invention decreases in the early stages of senescence, it can be effectively utilized in a biomarker composition for diagnosing senescence, a kit for diagnosing senescence including the same or a method of providing information for diagnosing senescence.

### DESCRIPTION OF DRAWINGS

FIG. 1a shows that the deregulation of mitoribosomal proteins (MRP) is a key event that occurs during the early (E-M) transition of replicative senescence (RS). It shows the expression heatmap of genes from MitoCarta3.0 in RS of HDF and unsupervised hierarchical clustering (metric=1−Pearson correlation, linkage method=average). DT and PT represent the doubling time (day) and the number of population doublings to reach cell conditions, respectively. E, M, A and VA represent cells in early, intermediate, advanced and very advanced senescence stages, respectively.

FIG. 1b shows that the deregulation of mitoribosomal proteins (MRP) is a key event that occurs during the early (E-M) transition of replicative senescence. It shows a principal component (PC) plot of RS that shows a clear separation between samples at different stages of senescence. E, M, A and VA represent cells in early, intermediate, advanced and very advanced senescence stages, respectively.

FIG. 1c shows that the deregulation of mitoribosomal proteins (MRP) is a key event that occurs during the early (E-M) transition of replicative senescence. It shows the expression patterns of M1 to M10 gene clusters identified by fuzzy c-means clustering for DT. The ellipse represents one standard deviation from the Gaussian mean fitted to the sample. n represents the number of genes assigned to each cluster.

FIG. 1d shows that the deregulation of mitoribosomal proteins (MRP) is a key event that occurs during the early (E-M) transition of replicative senescence. It shows the expression heatmap from RS-M1 to M3 gene cluster. MCD=mitochondrial central dogma; OXPHOS=oxidative phosphorylation; PISH=protein import, sorting and homeostasis; MDS=mitochondrial dynamics and surveillance; SMT=small molecule transport. E, M, A and VA represent cells in early, intermediate, advanced and very advanced senescence stages, respectively.

FIG. 1e shows that the deregulation of mitoribosomal proteins (MRP) is a key event that occurs during the early (E-M) transition of replicative senescence. It shows a bar chart and pie charts showing the number and proportion of genes assigned to each category defined by Mito-Carta3.0. The pie chart (bottom) shows the proportion of MRP or RP genes present in each gene cluster.

FIG. 1f shows that the deregulation of mitoribosomal proteins (MRP) is a key event that occurs during the early (E-M) transition of replicative senescence. It shows the expression heatmaps of MRPs and RPs. E, M, A and VA represent cells in early, intermediate, advanced and very advanced senescence stages, respectively.

FIG. 1g shows that the deregulation of mitoribosomal proteins (MRP) is a key event that occurs during the early (E-M) transition of replicative senescence. The expression patterns of cytoplasmic ribosomal proteins (RP, n=83) and mitochondrial ribosomal proteins (MRP, n=53) show progressive downregulation with respect to DT as identified by fuzzy c-means clustering. E, M, A and VA represent cells in early, intermediate, advanced and very advanced senescence stages, respectively.

FIG. 2 shows that fuzzy c-means clustering of the RS model using MRP and cytoplasmic RP identified 10 gene clusters. Clusters showing downregulated expression patterns in DT during the E to M transition were highlighted in red.

FIG. 3a shows SA-MPR, which is commonly downregulated in cellular senescence. It shows the expression heatmap of genes of MitoCarta 3.0 in HDF OS.

FIG. 3b shows SA-MPR, which is commonly downregulated in cellular senescence. Fuzzy c-means clustering of genes from MitoCarta 3.0 identified 4 gene clusters, termed M1 to M4.

FIG. 3c shows SA-MPR, which is commonly downregulated in cellular senescence. It shows a bar chart and pie charts showing the number and proportion of genes assigned to each category defined by MitoCarta 3.0.

FIG. 3d shows SA-MPR, which is commonly downregulated in cellular senescence. It shows a Venn diagram that shows the number of identified MRPs whose expression patterns were downregulated over time in each cellular senescence model. Gene symbols for MRPs that were commonly downregulated in both models are shown in yellow boxes. (E) The average gene expressions of commonly downregulated MRPs are shown for RS (left) and OS (right).

FIG. 4a shows that early MRP downregulation is associated with mitochondrial dysfunction and acquisition of a premature senescence phenotype. Replicative senescence (RS) was developed using native HDF. It shows a time series Western blot analysis of HDF-RS. Representative blot images are shown.

FIG. 4b shows that early MRP downregulation is associated with mitochondrial dysfunction and acquisition of a premature senescence phenotype. Replicative senescence (RS) was developed using native HDF. The increase in cell size of suspended cells was estimated by flow cytometry analysis of forward scatter (FSC). Representative FSC patterns are shown in the right panel. **, p<0.01 vs. DT2 by Student's t-test.

FIG. 4c shows that early MRP downregulation is associated with mitochondrial dysfunction and acquisition of a premature senescence phenotype. Replicative senescence (RS) was developed using native HDF. The increase in cell size was estimated by side scatter (SSC) analysis. Representative SSC patterns are shown in the right panel. **, p<0.01 vs. DT2 by Student's t-test.

FIG. 4d shows that early MRP downregulation is associated with mitochondrial dysfunction and acquisition of a premature senescence phenotype. Replicative senescence (RS) was developed using native HDF. It shows the time series of the primary HDF and Western blot of RS.

FIG. 4e shows that early MRP downregulation is associated with mitochondrial dysfunction and acquisition of a premature senescence phenotype. Replicative senescence (RS) was developed using native HDF. Mitochondrial ROS levels in cells were monitored by flow cytometry after MitoSox staining. Representative fluorescence shifts are shown (right panel). **, p<0.01 vs. DT2 by Student's t-test.

FIG. 4f shows that early MRP downregulation is associated with mitochondrial dysfunction and acquisition of a premature senescence phenotype. Replicative senescence (RS) was developed using native HDF. Representative images (top) and quantification (bottom) results for SA-β-gal activity are shown.

FIG. 4g shows that early MRP downregulation is associated with mitochondrial dysfunction and acquisition of a premature senescence phenotype. The HDF-OS model was developed by H₂O₂ treatment. Cellular responses to different doses of H₂O₂ are shown. Western blot results of the HDF-OS model are shown. Representative blot images are shown.

FIG. 4h shows that early MRP downregulation is associated with mitochondrial dysfunction and acquisition of a premature senescence phenotype. The HDF-OS model was developed by H₂O₂ treatment. It indicates SA-β-gal activity. **, p<0.01 vs C by Student's t-test.

FIG. 5 shows the quantification results of the time series MRP expression of the HDF-RS model of FIG. 4a. Quantified values of target protein levels in immunoblots were obtained by densitometric analysis using Image J. Actin values for each blot were used as controls. Heatmaps were generated using average values of MRP (top panel) and mitochondrial protein expression (bottom). Values are expressed as mean standard deviation (SD) (n=3). *, p<0.05; **, p<0.01 vs E (average of two DT2 values) by Student's t-test.

FIG. 6 shows the quantification results of MRP expression in dose response of the HDF-OSIS model in FIG. 4g. Quantified values of target protein levels in immunoblots were obtained by densitometric analysis using Image J. Actin values for each blot were used as controls. Heatmaps were generated using average values of MRP (top panel) and mitochondrial protein expression (bottom). Values are expressed as mean standard deviation (SD) (n=3). *, p<0.05; **, p<0.01 vs C by Student's t-test.

FIG. 7a shows that mitoribosome disruption induces mitochondrial ROS production and senescence. HDF (DT2) was treated with the indicated doses of chloramphenicol (CAP), which is a specific mitoribosome inhibitor. DMSO was used as vehicle (V) for CAP. Western blot results are shown. Representative blot images and quantified values are shown.

FIG. 7b shows that mitoribosome disruption induces mitochondrial ROS production and senescence. HDF (DT2) was treated with the indicated doses of chloramphenicol (CAP), which is a specific mitoribosome inhibitor. DMSO was used as vehicle (V) for CAP. Cell growth results are shown. **, p<0.01 vs. V (0.4%) by Student's t-test.

FIG. 7c shows that mitoribosome disruption induces mitochondrial ROS production and senescence. HDF (DT2) was treated with the indicated doses of chloramphenicol (CAP), which is a specific mitoribosome inhibitor. DMSO was used as vehicle (V) for CAP. It indicates SA-β-gal activity. **, p<0.01 vs. V (0.4%) by Student's t-test.

FIG. 7d shows that mitoribosome disruption induces mitochondrial ROS production and senescence. (A-D) HDFs (DT2) were treated with the indicated doses of chloramphenicol (CAP), which is a specific mitoribosome inhibitor. DMSO was used as vehicle (V) for CAP. Western blot results are shown. Representative blot images and quantified values are shown.

FIG. 7e shows that mitoribosome disruption induces mitochondrial ROS production and senescence. HDF (DT2) was individually transfected with siRNA against the indicated MRPs for 4 days. For the negative control (NC), siRNA with a random sequence was used. Western blot results are shown. Representative blot images and quantified values are shown.

FIG. 7f shows that mitoribosome disruption induces mitochondrial ROS production and senescence. HDF (DT2) was individually transfected with siRNA against the indicated MRPs for 4 days. For the negative control (NC), siRNA with a random sequence was used. (F) SA-β-gal activity, (G) cell growth, (H) cell size (FSC), and (I) cell granularity (SSC). **, p<0.01 vs. NC by Student's t-test.

FIG. 7g shows that mitoribosome disruption induces mitochondrial ROS production and senescence. HDF (DT2) was individually transfected with siRNA against the indicated MRPs for 4 days. For the negative control (NC), siRNA with a random sequence was used. Results of mitochondrial ROS (MitoSOX staining) are shown. **, p<0.01 vs. NC by Student's t-test.

FIG. 7h shows that mitoribosome disruption induces mitochondrial ROS production and senescence. HDF (DT2) was individually transfected with siRNA against the indicated MRPs for 4 days. For the negative control (NC), siRNA with a random sequence was used. It indicates mitochondrial translation activity. In order to selectively monitor mitochondrial translation activity, emetine (a cytosolic translation inhibitor) and homopropargylglycine (HPG—a tracer of nascent protein synthesis) were used. Representative images of protein translation activity for total, cytosolic (Cyto) and mitochondria (Mito) are shown in the left panel, and selective mitochondrial translation activity after individual knockdown of the three MRPs is shown in the right panel.

FIG. 8a shows that TPP1 expression decreases in the M phase of RS, and its inhibition induces senescence with telomeric DNA damage. This is the result of estimating the relative telomere length of HDF in the early (E) and middle (M) stages of HDF-RS. **, p<0.01 vs DT2 by Student's t-test.

FIG. 8b shows that TPP1 expression decreases in the M phase of RS, and its inhibition induces senescence with telomeric DNA damage. It shows a schematic structure of the shelterin complex.

FIG. 8c shows that TPP1 expression decreases in the M phase of RS, and its inhibition induces senescence with telomeric DNA damage. Expression heatmaps of TERT (telomerase) and shelterin complex genes in RS and OSIS were obtained from previous time series transcriptome data (GSE41714 and GSE80322).

FIG. 8d shows that TPP1 expression decreases in the M phase of RS, and its inhibition induces senescence with telomeric DNA damage. It shows messenger RNA levels of HDFs at the indicated DTs of the RS model by qPCR analysis. HDFs with two different cell population doublings (PD32 and PD35) were used for DT2. **, p<0.01 and *, p<0.05 vs. DT2 (PD32) by Student's t-test.

FIG. 8e shows that TPP1 expression decreases in the M phase of RS, and its inhibition induces senescence with telomeric DNA damage. It shows a Western blot of time series HDF-RS. Representative blot images and quantified values are shown.

FIG. 8f shows that TPP1 expression decreases in the M phase of RS, and its inhibition induces senescence with telomeric DNA damage. HDFs (DT2) were individually transfected with siRNA against target genes for 4 days. Western blot results are shown. Representative blot images and quantified values are shown.

FIG. 8g shows that TPP1 expression decreases in the M phase of RS, and its inhibition induces senescence with telomeric DNA damage. HDFs (DT2) were individually transfected with siRNA against target genes for 4 days. It indicates SA-β-gal activity. **, p<0.01 and *, p<0.05 vs. NC by Student's t-test.

FIG. 8h shows that TPP1 expression decreases in the M phase of RS, and its inhibition induces senescence with telomeric DNA damage. HDFs (DT2) were individually transfected with siRNA against target genes for 4 days. Telomere length analysis is shown.

FIG. 8i shows that TPP1 expression decreases in the M phase of RS, and its inhibition induces senescence with telomeric DNA damage. HDFs (DT2) were individually transfected with siRNA against target genes for 4 days. Western blot results are shown. Representative blot images and quantified values are shown.

FIG. 8j shows that TPP1 expression decreases in the M phase of RS, and its inhibition induces senescence with telomeric DNA damage. HDFs (DT2) were individually transfected with siRNA against target genes for 4 days. Telomere dysfunction-induced foci (TIFs), colocalized foci of telomeres (green) and 53BP1 (red) were visualized by IF-FISH. Nuclei were visualized using DAPI (blue) staining.

FIG. 9 shows the quantification results of TIF after knockdown of target genes. TIF quantification was obtained from the IF-FISH results in FIG. 8j (A) and FIG. 10f (B). TIF positive (+) cells were selected for cells with three or more TIFs in the nucleus. Values for TIF (+) cells were obtained by counting at least 20 cells in 5 images from 3 independent labeling slips. p<0.01 vs. Student's t-test. N.C.

FIG. 10a shows that SA-MRP deregulation acts as an upstream regulator of TPP1 inhibition. HDF (DT2) was transfected with siRNA against targets (TPP1 and POT1) for 4 days. Western blot results are shown. Representative blot images and quantified values are shown. Arrows indicate POT1 bands.

FIG. 10b shows that SA-MRP deregulation acts as an upstream regulator of TPP1 inhibition. HDF (DT2) was transfected with siRNA against targets (TPP1 and POT1) for 4 days. Messenger RNA levels by qPCR are shown 4 days after individual knockdown of TPP1 and POT1. **, p<0.01 vs NC by Student's t-test.

FIG. 10c shows that SA-MRP deregulation acts as an upstream regulator of TPP1 inhibition. HDF (DT2) were transfected with siRNA against the target MRP for 4 days. Western blot results are shown. Representative blot images and quantified values are shown. Arrows indicate POT1 bands.

FIG. 10d shows that SA-MRP deregulation acts as an upstream regulator of TPP1 inhibition. Messenger RNA levels (qPCR) are shown 4 days after individual knockdown of MRPS9, MRPS15 and MRPS31. **, p<0.01 and *, p<0.05 vs. NC by Student's t-test. HDF (DT2) were transfected with siRNA against the target MRP for 4 days.

FIG. 10e shows that SA-MRP deregulation acts as an upstream regulator of TPP1 inhibition. It indicates relative telomere length. HDF (DT2) was transfected with siRNA against the target MRP for 4 days.

FIG. 10f shows that SA-MRP deregulation acts as an upstream regulator of TPP1 inhibition. HDF (DT2) was transfected with siRNA against the target MRP for 4 days. Telomere dysfunction-induced foci (TIFs), which are foci of colocalization of telomeres (green) and 53BP1 (red), were visualized by IF-FISH as described in Materials and Methods. Nuclei were visualized using DAPI (blue) staining.

FIG. 10g shows that SA-MRP deregulation acts as an upstream regulator of TPP1 inhibition. HDF (DT2) was transfected with siRNA against the target MRP for 4 days. Western blot results are shown. Representative blot images and quantified values are shown.

FIG. 10h shows that SA-MRP deregulation acts as an upstream regulator of TPP1 inhibition. HDF (DT2) was exposed to CAP (200 μg/mL) for the indicated periods. Western blot results are shown. Representative blot images and quantified values are shown. Arrows indicate POT1 bands.

FIG. 10i shows that SA-MRP deregulation acts as an upstream regulator of TPP1 repression. HDF (DT2) was exposed to CAP (200 μg/mL) for the indicated periods. Quantitative RT-PCR results are shown. **, p<0.01 and *, p<0.05 vs. NC by Student's t-test.

FIG. 10j shows that SA-MRP deregulation acts as an upstream regulator of TPP1 inhibition. HDF (DT2) was exposed to CAP (200 μg/mL) for the indicated periods. Western blot analysis after HDFs (M stage, PD63 and DT3) were transfected with pCMV6-ACD-AC-GFP plasmid for 2 days. Representative blot images and quantified values are shown. Arrows indicate POT1 bands.

FIG. 11a shows the verification results of SA-MRP deregulation in aged human cells. It shows a heatmap of the expression of SA-MRP and shelterin genes in human primary skin fibroblasts. There are two POT1 orthologs, Pot1a and pot1b, in the mouse genome.

FIG. 11b shows the verification results of SA-MRP deregulation in aged mouse skin tissue. It shows a heatmap of the expression of SA-MRP and shelterin genes in mouse dermal fibroblasts. There are two POT1 orthologs, Pot1a and pot1b, in the mouse genome.

FIG. 11c shows the verification results of SA-MRP deregulation in aged mouse skin tissue. It shows an expression heatmap of SA-MRP and shelterin genes in mouse skin tissue. There are two POT1 orthologs, Pot1a and pot1b, in the mouse genome. Welch t-test p-values are shown in each right panel.

FIG. 11d shows the verification results of SA-MRP deregulation in aged human cells and aged mouse/rat skin tissue. It shows a Venn diagram that shows the number of identified MRPs with downregulated expression patterns in aged samples in each data set analyzed. Gene symbols of the 15 shared SA-MRPs in all human and mouse cells and tissues used are shown in boxes.

FIG. 11e shows the verification results of SA-MRP deregulation in aged human cells and aged mouse/rat skin tissue. Messenger RNA levels (qPCR) of target genes using aged mouse skin tissue are shown. **, p<0.01 and *, p<0.05 vs. 4m by Student's t-test.

FIG. 11f shows the verification results of SA-MRP deregulation in aged human cells and aged mouse/rat skin tissue. Messenger RNA levels (qPCR) of target genes using aged mouse skin tissue are shown. *, p<0.05 vs. 6m by Student's t-test.

FIG. 12a shows that single cell RNA-seq analysis identifies distinct cell clusters with unique expression profiles of senescence-related MRP and shelterin genes. It shows the identification of highly variable features in scRNA-seq data derived from mouse dermal fibroblasts (GSE111136).

FIG. 12b shows that single cell RNA-seq analysis identifies distinct cell clusters with unique expression profiles of senescence-related MRP and shelterin genes. The distribution of p-values for each PC is shown, with a uniform distribution (dotted line) representing significant PCs.

FIG. 12c shows that single cell RNA-seq analysis identifies distinct cell clusters with unique expression profiles of senescence-related MRP and shelterin genes. Uniform manifold approximation and projection (UMAP) plots depicting cells of different Seurat-defined cell clusters (left) and ages (right) are shown.

FIG. 12d shows that single cell RNA-seq analysis identifies distinct cell clusters with unique expression profiles of senescence-related MRP and shelterin genes. It shows a stacked bar chart that shows the number and percentage of cells assigned to Seurat-defined cell clusters.

FIG. 12e shows that single cell RNA-seq analysis identifies distinct cell clusters with unique expression profiles of senescence-related MRP and shelterin genes. Expression heatmaps of the top 10 genes for each sample (young and old) only in C3 (top) and C4 cells (bottom) are shown.

FIG. 12f shows that single cell RNA-seq analysis identifies distinct cell clusters with unique expression profiles of senescence-related MRP and shelterin genes. It shows a dot plot showing the top GO terms enriched in each cell group.

FIG. 12g shows that single cell RNA-seq analysis identifies distinct cell clusters with unique expression profiles of senescence-related MRP and shelterin genes. It shows (G) an expression heatmap and (H) mean expression (SD) of 47 mouse SA-MRPs, 15 SA-MRP genes, shelterin complex, Acd and senescence markers (Cdkn1a for p21 and Cdkn2a for p16). Kruskal-Wallis chi-square p-value (p) is displayed. N represents the number of genes analyzed. Among the 51 SA-MRPs, Mrps16, Mrps37, Mrps30 and Mrps27 are absent in the filtered data set.

### MODES OF THE INVENTION

### Experiment Method

### 1. Transcriptome Analysis of Cellular Senescence Model

Processed transcriptome data derived from the replicative senescence (RS) model of human primary fibroblasts generated by [Non-Patent Document 1] and [Non-Patent Document 2] and the oxidative stress-induced senescence (OS) model induced by hydrogen peroxide treatment were downloaded from the NCBI Gene Expression Omnibus (GEO) under the accession codes GSE41714 and GSE80322, respectively. Probe IDs were mapped to gene symbols using the mapIds function in the R AnnotationDbi package (v1.52.0). Probes with the highest average expression level across samples were collapsed into genes for subsequent analysis. Principal component analysis (PCA) was performed using the prcomp function in the R stats package (v4.0.3). Fuzzy c-means clustering was applied to the scaled data with a predetermined number of clusters (n=10 for RS, n=4 for OS) using the mfuzz function of the R Mfuzz package (v2.50.0).

### 2. MitoCarta3.0 and Genes Encoding MRP and RP

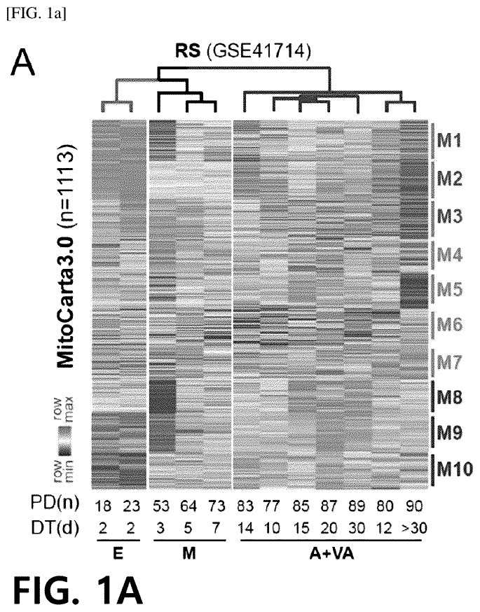

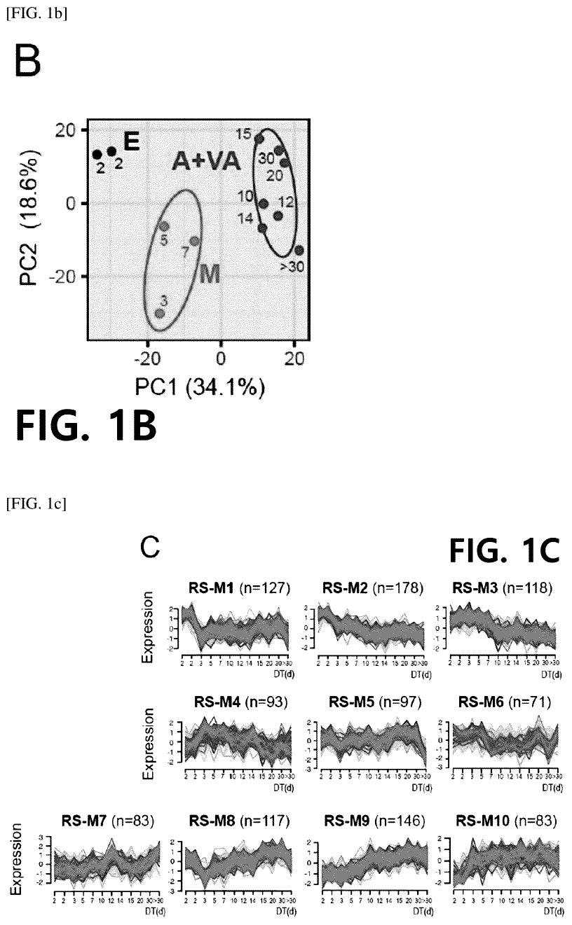

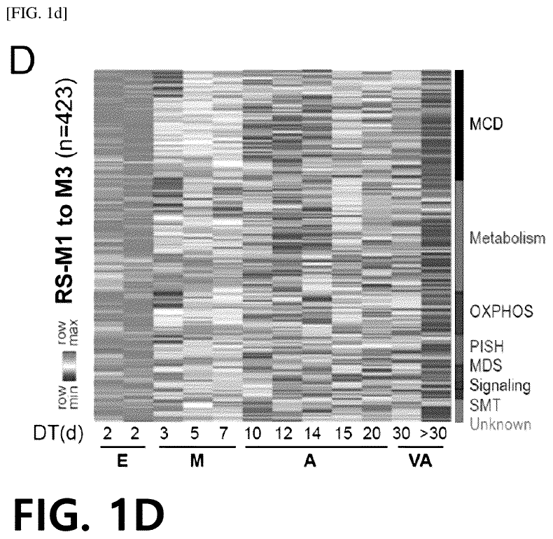

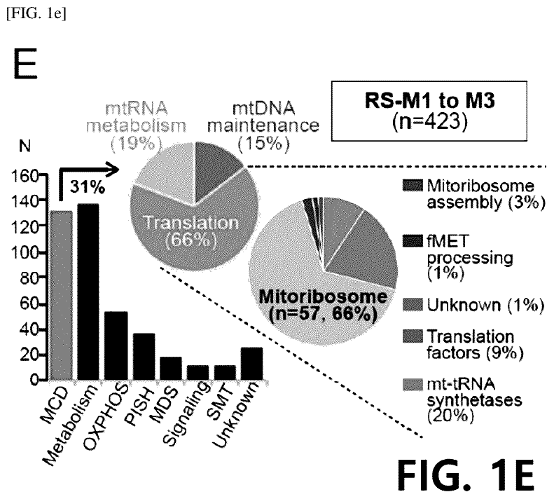

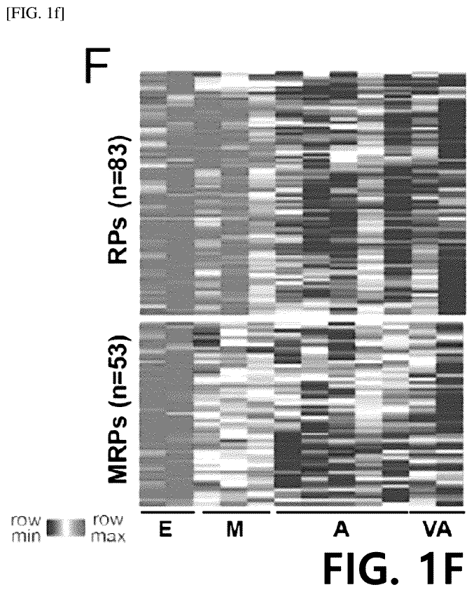

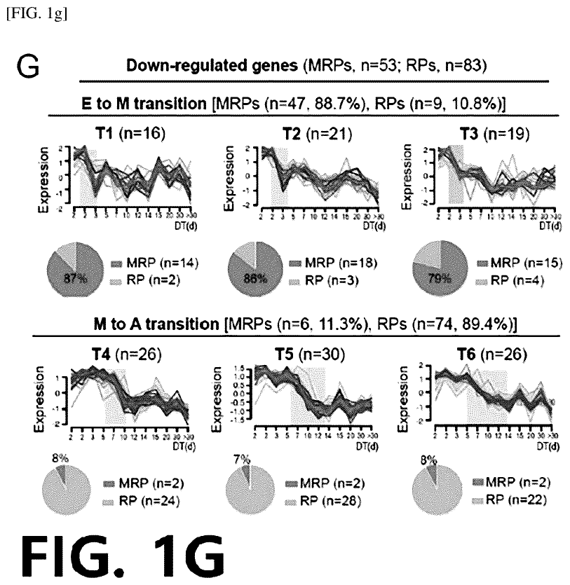

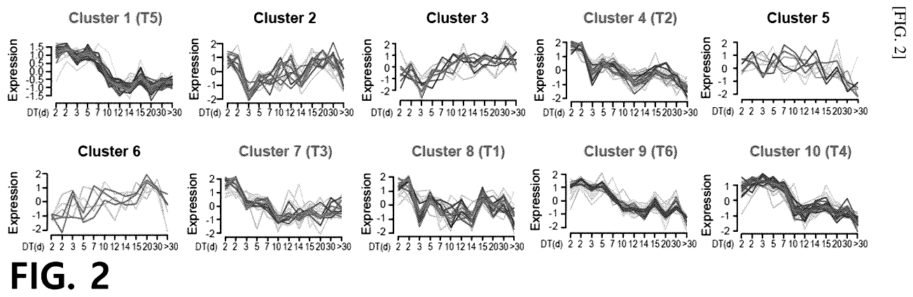

Human gene annotation of the mitochondrial proteome, including sub-mitochondrial compartments and pathways, was directly obtained from MitoCarta3.0 (https://www.broadinstitute.org/mitocarta/mitocarta30-inventory-mammalian-mitochondrial-proteinsand-pathways, last accessed on Apr. 5, 2021). Transcriptome data for 1,113 of the 1,136 genes listed in MitoCarta3.0 are available on the Illumina HumanHT-12 v4.0 Expression BeadChip (GPL10558), which is a profiling platform used to generate both data sets. Fuzzy c-means clustering of the RS model was performed by using genes encoding MRPs (FIGS. 1f, g and FIG. 2), and the list of genes encoding proteins of small (n=30) and large (n=52) ribosomal subunits was obtained from Ban Lab (https://bangroup.ethz.ch/research/nomenclature-of-ribosomal-proteins.html, last accessed on Apr. 5, 2021). A list of genes encoding cytoplasmic ribosomal proteins (RPs) was directly obtained from the HUGO Gene Nomenclature Committee (HGNC) resource, https://www.genenames.org/data/genegroup/#!/group/646, last accessed on Apr. 5, 2021).

### 3. Transcriptome Analysis of Senescence Skin in Mice and Humans

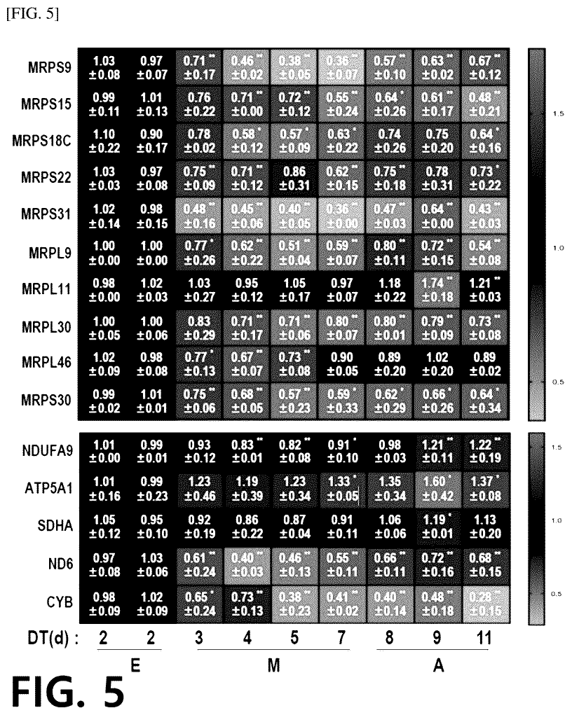

RPKM (reads per kilobase of transcript per million mapped reads) values derived from mouse skin at five different ages were downloaded directly from NCBI GEO with access code GSE75192. Processed expression matrices of dermal fibroblasts isolated from young (n=4, 2 months) and old (n=4, 18 months) mice were obtained by using the getGeneExpressionFromGEO function in the R geneExpressionFrom-GEO package (v0.6). For human transcriptome data, quartile-normalized expression data for primary dermal fibroblasts from Caucasian men (n=5, median age=24) and older adults (n=5, median age=70.4) were directly downloaded from NCBI GEO with the accession code GSE131938. Using Morpheus (https://software.broadinstitute.org/morpheus/, last accessed on Apr. 5, 2022), the representational heatmaps of MRPs were generated and hierarchically clustered using 1-Pearson correlation distance and average linkage to determine downregulated MPRs. Senescence for the three analyzed data sets (FIG. 5). Human gene symbols were converted to mouse symbols using the R biomaRt package (v2.46.3).

### 4. Single-Cell RNA-Seq Analysis of Mouse Senescence Skin Fibroblasts

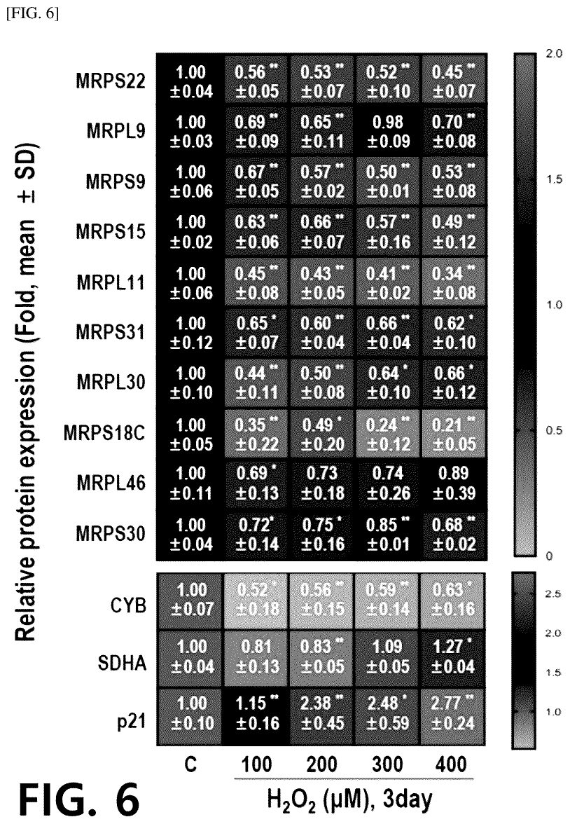

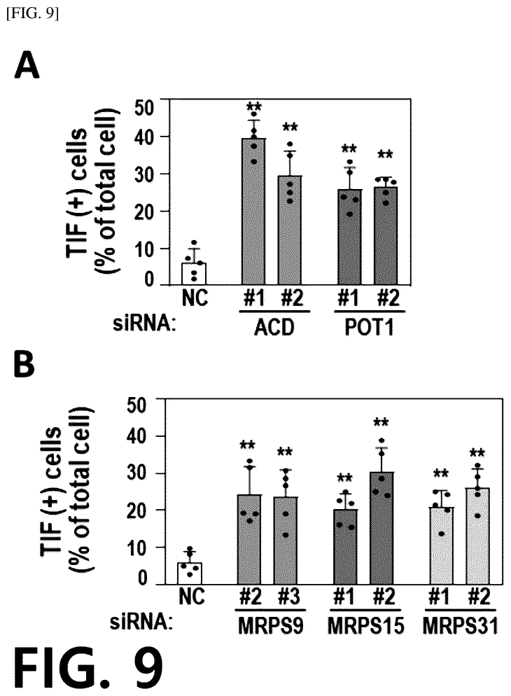

Processed expression matrices with raw read counts per cell index of dermal fibroblasts obtained from mice at three ages (1.5/2.5 days old (newborn), 2 months old (young) and 18 months old (old)) were downloaded from NCBI GEO according to the accession code GSE111136. The R Seurat package (v4.0.1) was used to perform normalization, feature selection, data size scaling, PCA, cell clustering, nonlinear dimensionality reduction (e.g., Uniform Manifold approximation and projection (UMAP) plotting) and heatmap generation. Briefly, the most variable genes (n=3,000) were used to perform PCA on the scaled data, and herein, the first 10 PCs were used as input for cell clustering. Clusters of cells predominantly expressing mitochondrial genes were excluded for further analysis (FIG. 6). Cells from the C3 and C4 clusters were extracted to generate independent Seurat entities, which were used to identify differentially expressed (DE) features in fibroblasts from young and old mice using the FindAllMarkers function. Gene ontology (GO) enrichment analysis was performed by using the richGO function of the R clusterProfiler package (v3.18.1) using the top 100 DE genes (FIG. 9).

### 5. Cell Culture, Cell Growth and Cell Senescence Development

Primary HDF isolated from the foreskin of a 5-year-old boy was provided by Dr. Lim IK (Ajou University) and incubated in DMEM (Antibiotic-Antimycotic™ Grand Island, NY, USA) supplemented with 10% FBS (Gibco, Grand Island, NY, USA) and antibiotics at 37° C. in a humidified incubator with 5% CO₂ as previously described. Cell growth and viability were monitored by trypan blue staining. At the end of each experiment, cells were harvested by trypsin treatment, stained with 0.4% (w/v) trypan blue (Invitrogen) to exclude dead cells, and then counted using a Countess™ automatic cell counter (Invitrogen, Carlsbad, CA, USA).

In order to develop RS, confluent HDFs were transferred evenly into two new dishes and sub-cultured sequentially, and population doubling (PD) number and doubling time (DT) were continuously monitored as previously described. The obtained DT values included the adaptation time after HDF was spread on a new plate. In order to generate the OS of HDFs, primary HDFs (DT2) were exposed to H₂O₂ as previously described.

### 6. Skin Tissue of Senescence Animals

Young (4 months, n=3) and old (12-18 months, n=3) male C57BL/6 mice were purchased from KBSI (Korea Basic Science Institute, Deajeon, Korea). After sacrifice, skin tissue was collected and stored at 80° C. All animal experiments were approved by the Animal Research Ethics Committee of Ajou University Medical Center (approval number: 2020-0051). Skin tissues from young (6 months old, n=3) and old (15 and 24 months old, n=3) Sprague Dawley (SD) rats were provided by the Senescence Tissue Bank, Busan, Korea.

### 7. Measurement of Mitochondrial ROS Levels and Monitoring Cell Size and Cell Granularity

In order to determine mitochondrial ROS levels, cells were incubated in medium containing 10 μM Mitosox fluorescent probe (Invitrogen) for 15 minutes at 37° C. Stained cells were washed, resuspended in PBS and analyzed by flow cytometry (FACS Vantage, Becton Dickinson Corp., San Antonio, TX, USA). The average of arbitrary fluorescence units from 10,000 cells was obtained.

### 8. SA-β-Gal Activity Staining

Cells seeded in 12-well plates were subjected to SA-β-gal activity staining. An increase in SA-β-gal activity was monitored using the SA-β-gal staining kit (CST, #9860S, Danvers, MA, USA) according to the instructions provided. Representative images of each well were photographed using a Leica DMi1 inverted microscope (Leica Microsystems, Wetzlar, Germany). The percentage of positive (+) cells for SA-β-gal was obtained by counting the total number of blue-stained cells using Image J software (National Institutes of Health, https://imagej.nih.gov/ij/, last accessed May 20, 2022). For each experimental condition, three wells were used and two independent experiments were performed (total n=6).

9. Transfection of siRNA and Expression Plasmid into Cells

HDFs were transfected with siRNA duplexes against target genes using Lipofectamine RNAiMAX™ reagent (Invitrogen, Carlsbad, CA, USA) according to the provided instructions. Target siRNA duplexes were generated by Bioneer (Daejeon, Korea), and the nucleotide sequences are as follows.

TABLE 1

| | | SEQ |
|---|---|---|
| Target | Sequence | ID NO: |
| MRPS9 | #1: 5′-GAGGUUUCGAAGAAGUGUA | 1 |
| | #2: 5′-GAAAACUUACACAGAGGAUU | 2 |
| MRPS15 | #1: 5′-CACUUGGAGAAACAUCGAA | 3 |
| | #2: 5′-GUCUGUCAAGAUCCGCAGU | 4 |
| MRPS31 | #1: 5′-CUCGGCGAUGUUUCCUAGA | 5 |
| | #2: 5′-GAUUCUCUCCCUUUUGAUA | 6 |
| ACD | #1: 5′-GACCUAUCUCUGACCCUCA | 7 |
| | #2: 5′-CCUACCGGUACCACAGGAA | 8 |
| POT1 | #1: 5′-CUGAUGUGGAUCAACUGAA | 9 |
| | #2: 5′-CUGAUCAAGCUACAGUGUA | 10 |
| Negative | 5′-CCUACGCCACCAAUUUGGU | 11 |
| Control | | |

The TPP1 overexpression plasmid (pCMV6-ACD-AC-GFP) was purchased from OriGene Technologies (Rockville, MD, USA) and introduced into HDFs using Fugene® Reagent (Promega, Fitchburg, WI, USA).

### 10. Western Blot Analysis

Cells were washed twice with PBS and lysed in RIPA lysis buffer (150 mM NaCl, 1% NP-40, 0.5% sodium deoxycholate, 0.1% SDS, 50 mM Tris (pH 8.0), 1 mM NaF, 1 mM Na3VO4, 1mMPMSF, 1.5 μg/mL. mL pepstatin A, 1.5 μg/mL leupeptin). MRPS9 (GTX87450), MRPS18C (GTX87389), MRPS22 (GTX45001), MRPS30 (GTX54320), MRPL9 (GTX116779), MRPL46 (GTX87311), MT to MRPL46 (GTX0) and Telomerease (GTX30410) were purchased from GeneTex. Inc. (Irvine, CA, USA). Antibodies against MRPS15 (ab137070), MRPS31 (ab167406), MRPL30 (ab179819) and POT1 (ab124784) were purchased from Abcam (Cambridge, MA, USA). Antibodies against MRPL11 (2199S) and TPP1 (14667S) were obtained from Cell Signaling Technology (Danvers, MA, USA), and antibodies against NDUFA9 (A21344), ATP5A1 (A21350), SDHA (A11142) and MT-ND6 (A3185) were obtained from Invitrogen (Carlsbad, CA, USA). Antibodies against p21 (sc-6246) and β-Actin were purchased from Santa Cruz (Dallas, TX, USA). β-Actin was used as a loading control to obtain immunoblots for various target proteins using the same cell lysate. Multiple blots were cut into two or three pieces for hybridization with different target antibodies and re-probed for rechecking if necessary. Representative blots shown in the results were selected from results in which actin levels were comparable. At least 4 independent experiments were performed for each target protein. Quantitative values of target protein levels on immunoblots were obtained by densitometric analysis using Image J (National Institutes of Health, https://imagej.nih.gov/ij/, last accessed on May 20, 2022).

11. Quantitative Real-Time PCR (qPCR) for mRNA Expression

Total cellular RNA was isolated using RNAisolation plus kit (MN, Dueren, Germany), and cDNA was prepared using ReverTra Ace™ RT Master Mix (Toyobo Co. Ltd., Osaka, Japan). PCR was performed using GoTaq® Master Mix (Promega, Fitchburg, WI, USA) according to the provided protocol. The targeting primer set was produced by Macrogen (Seoul, Korea).

In order to isolate total RNA from animal skin tissue, the Maxwell RSC Simply RNA Tissue kit (Promega, Fitchburg, WI, USA) was used according to the manufacturer's protocol. The sequences of the primer sets used are listed below.

TABLE 2

| Target | Species | | Primer Sequence | SEQ ID NO: |
|---|---|---|---|---|
| MRPS9 | Human | Forward | 5′-ATCACACAGGACAGAGAACAG | 12 |
| | | Reverse | 5′-ATTGCCAGTCGTATTGCTCC | 13 |
| MRPS15 | | Forward | 5′-AGCGCCAAGTTTCCTTTCAAC | 14 |
| | | Reverse | 5′-CTTTGAGCAGCGTAGAGGGA | 15 |
| MRPS31 | | Forward | 5′-GACGTTCCTACCTCTTCGCC | 16 |
| | | Reverse | 5′-CTGCGGTACCTGACTGTTCC | 17 |
| ACD | | Forward | 5′-TTGAGGTACTACAGGACGCCG | 18 |
| | | Reverse | 5′-CGAACTCCTTCTCCTCCCAGT | 19 |
| POT1 | | Forward | 5′-GCCTTACGTGTTTGGGCATC | 20 |
| | | Reverse | 5′-TGGTGCCATCCCATACCTTT | 21 |
| TERF1 | | Forward | 5′-CAGCGCAGAGGCTATTATTC | 22 |
| | | Reverse | 5′-TCAAACTGTGCATCAAGGG | 23 |
| TINF2 | | Forward | 5′-CAGGGGGAAGGAAAGGAATC | 24 |
| | | Reverse | 5′-GAGGCAGGAGACTAGAGTAC | 25 |
| TERF2 | | Forward | 5′-TCGAGAAAAGAACTTGGCCC | 26 |
| | | Reverse | 5′-CCTTTTTGGCCATCGTGAGG | 27 |
| TERF2IP | | Forward | 5′-GACGTTCCTACCTCTTCGCC | 28 |
| | | Reverse | 5′-CTGCGGTACCTGACTGTTCC | 29 |
| TBP | | Forward | 5′-CGGATAGCGGGGAACCAC | 30 |
| | | Reverse | 5′-CACAAGCATCTTTTTGACTGC | 31 |
| Mrps7 | Mouse | Forward | 5′-AACCTTCCAGGGCTCACAC | 32 |
| | | Reverse | 5′-GCTCCTGGTCATACTTCTCC | 33 |
| Mrps18b | | Forward | 5′-CGCTTCTAAAGCTGGTTCC | 34 |
| | | Reverse | 5′-AGCGGTTGTGGTATTCTTC | 35 |
| Mrps26 | | Forward | 5′-CCTGCCAAATCCAAGGTCG | 36 |
| | | Reverse | 5′-CCAACGTGAACTCTAGCCTG | 37 |
| Mrps33 | | Forward | 5′-CGAGGTCAGGAGCAGGAATC | 38 |
| | | Reverse | 5′-AAACAGGCTCACCACTTTC | 39 |
| Acd | | Forward | 5′-TCCAAACTGCCAGGCTC | 40 |
| | | Reverse | 5′-TCAGCCCATGTGTCCTC | 41 |
| Tbp | | Forward | 5′-GTGGATCGAGTCCGGTAGC | 42 |
| | | Reverse | 5′-TGAAATAGTGATGCTGGGC | 43 |
| Mrps7 | Rat | Forward | 5′-GAACTCACCGAAGAGGAGAAG | 44 |
| | | Reverse | 5′-ATGTTGGTGAATTTGCTGATGAC | 45 |
| Mrps18b | | Forward | 5′-CGAAAGACATGCATTCGTAAGG | 46 |
| | | Reverse | 5′-GGCATGGAAGATGATACCCG | 47 |
| Mrps26 | | Forward | 5′-ACTGCAGGAACTACGGATAG | 48 |
| | | Reverse | 5′-TCTTTCAGTTGCACCCAAGC | 49 |
| Mrps33 | | Forward | 5′-GAGTGCCCGTATCTTTGGTG | 50 |
| | | Reverse | 5′-GTCCTGATGCTCATCTCTGTAA | 51 |
| Acd | | Forward | 5′-CCCACTCTGTCTGGGAACC | 52 |
| | | Reverse | 5′-GTTTGGACCTGGGTATGGAG | 53 |
| Tbp | | Forward | 5′-AATATAATCCCAAGCGGTTTG | 54 |
| | | Reverse | 5′-GCTGCTAGTCTGGATTGTTC | 55 |

### 12. Telomere Length Estimation

Telomere length was estimated by qPCR on genomic DNA with some modifications. Total cellular genomic DNA was isolated using routine methods. In order to estimate telomere length, qPCR was performed using GoTaq® Master Mix (Promega, Fitchburg, WI, USA). The primer set was manufactured by Macrogen (Seoul, Korea). The acidic ribosomal phosphoprotein P0 (36B4) gene, which is a known single copy gene, was used as an internal control. The sequences of the primer sets used are as follows.

TABLE 3

| Target | Sequence | SEQ ID NO: |
|---|---|---|
| Telomere | 5′-CGGTTTGTTTGGGTTTGGGTTTGGGTTTGGGTTTGGGTT | 56 |
| | 5′-GGCTTGCCTTACCCTTACCCTTACTTACCCTTACCCT | 57 |
| 36B4 | 5′-CAGCAAGTGGGAAGGTGTAATCC | 58 |
| | 5′-CCCATTCTATCATCAACGGGTACAA | 59 |

### 13. Monitoring Mitochondrial Translation Activity

Mitochondrial translation activity was monitored using the Click-iT™ HPG Alexa Fluor™ 488 Protein synthesis Assay Kit (Invitrogen, Carlsbad, CA, USA). In order to label newly synthesized proteins in mitoribosomes, cells were preincubated with 25 μg/mL emetine (Merck Millipore, Burlington, MA, USA) for 2 hours to inhibit cytoplasmic translation activity. Afterwards, cells were incubated for 1 hour in methionine-free RPMI medium (Invitrogen) supplemented with 50 μM L-homopropargylglycine (Click-iT™ HPG, Invitrogen). After washing twice with PBS, cells were lysed in lysis buffer (1% SDS, 50 mM Tris, pH 8.0, 1 mM NaF, 1 mM Na3VO4, 1 mM PMSF, 1.5 g/mL pepstatin A, 1.5 μg/mL leupeptin). Whole cell lysates (50 μg) were further conjugated with Alexa Fluor 488 in the Click-iT™ reaction cocktail for 30 minutes according to the manufacturer's protocol and subjected to Western blot analysis using Alexa Fluor 488 antibody (A-11094, Invitrogen). The specificity of mitochondrial translation activity was confirmed using chloramphenicol (Sigma, St. Louis, MO, USA).

### 14. Immunofluorescence-Fluorescence In-Situ Hybridization (IF-FISH) Analysis for Telomeric DNA Damage

In order to visualize telomeric DNA damage, cells grown on coverslips were first treated with 4% paraformaldehyde (for fixation), and were subjected to conventional immunofluorescence staining by using primary 53BP1 antibody (4937S, Cell signaling Technology, Danvers, MA, USA) and TRITC conjugated anti-rabbit secondary antibody (A16101, Invitrogen). After staining, cells were FISH-stained again according to the provided protocol with FITC-labeled telomere-specific (TTAGGG) PNA probe (PANAGENE, Daejeon, Korea). Nuclei were counterstained with 1 g/mL DAPI (D3571, Invitrogen, Carlsbad, CA, USA). Fluorescently stained cells were visualized with a Nikon A1R Spectral Confocal Laser Dual Scanning Microscope (Nikon Instruments Inc., Melville, NY, USA).

### 15. Statistical Analysis

Statistical analysis of the transcriptome was performed using R (v4.0.3) as described above, and other experimental data were evaluated by unpaired two-sample Student's t-test using GraphPad Prism software (La Jolla, CA, USA).

### Example 1

### Deregulation of Mitoribosome Genes, an Early Event in Replicative Senescence.

In order to investigate how mitochondrial defects may be involved in the replicative senescence (RS) process, the inventors of the present invention used time-series transcriptomes of RS from human diploid fibroblasts (HDFs) that were previously generated by the inventors of the present invention to analyze the expression profile of mitochondria-associated genes (MAG), which encode mitochondrial proteins listed in MitoCarta 3.0. In [Non-Patent Document 1], HDFs undergoing 4 stages of senescence, defined as early (E), intermediate (M), late (A) and very late (VA), with distinct gene expression patterns, were clustered into 3 groups based on MAG as expression profiles in which cells of A and VA stages were clustered together (FIGS. 1a, b). These data indicated that the major deregulation of MAG was completed before entering the A stage.

Fuzzy c-means clustering of the RS model using 1113 MAG identified 10 gene clusters (RS-M1 to M10) with distinct time-dependent expression patterns (FIG. 1c). In order to identify causal MAGs associated with mitochondrial defects related to senescence progression, the inventors of the present invention analyzed 3 clusters showing progressive downregulation of RS from E to VA stages, denoted RS-M1 to M3 (38.0% of 1,113 genes, FIG. 1d). The RS-M1 to M3 clusters mainly consisted of genes classified as mitochondrial central dogma (MCD) and metabolism according to MitoCarta3.0, where 57 genes were found to encode MRPs (FIG. 1e).

Fuzzy c-means clustering of the RS model using MRP and cytoplasmic ribosomal proteins (RP) identified 10 additional gene clusters, of which 6 clusters showed a downregulated expression pattern across doubling times (DT) (FIG. 2). In particular, most downregulation of MRPs occurred during the E to M transition (88.7%, 47 vs. 53 MRPs), whereas most downregulation of RPs was observed at later time points (89.4%, 74 vs. 83 RPs) (FIGS. 1f, g). These results indicate the occurrence of mitoribosome defects at the early E-M transition prior to cytoplasmic ribosome defects during the RS process in HDFs.

### Example 2

### Identification of Senescence-Related Mitoribosome Gene Signatures

In order to assess whether MRP deregulation may represent a general event of cellular senescence, the inventors of the present invention additionally analyzed the expression profile of MAG derived from time-series transcriptome data for an oxidative stress-induced senescence (OS) model of HDFs that were previously generated by the inventors of the present invention. As observed in the RS model, fuzzy c-means clustering of MAG identified 4 distinct gene clusters.

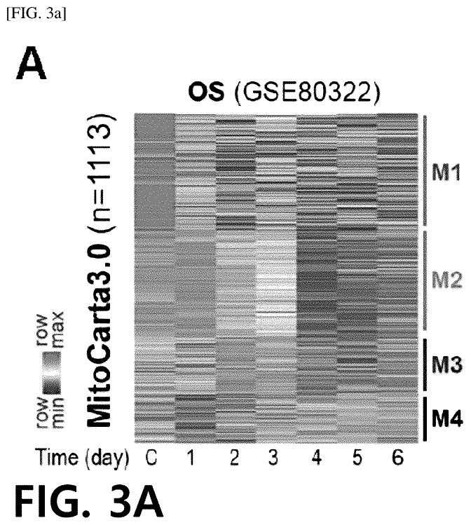

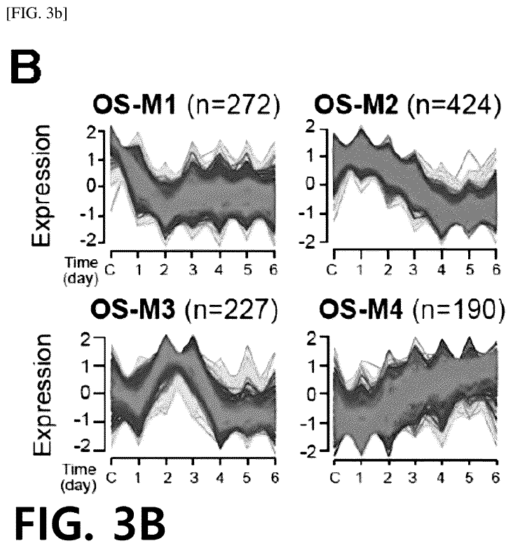

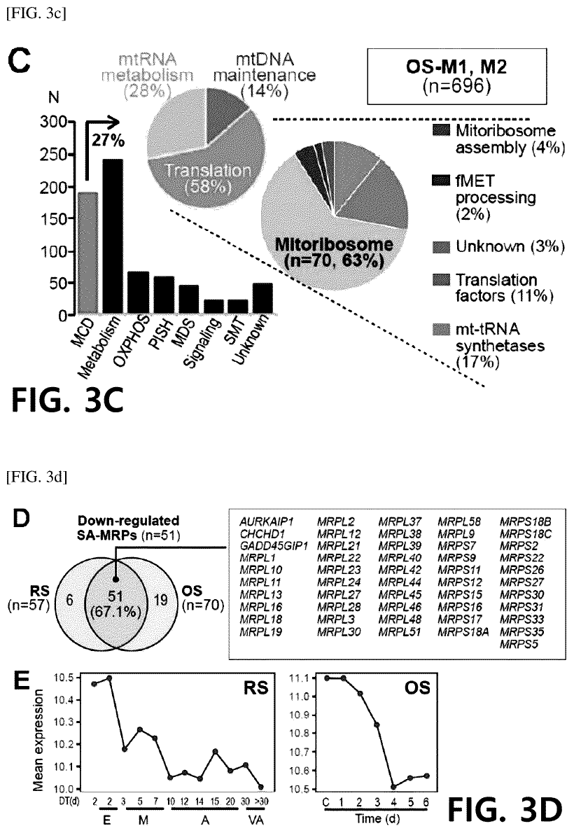

Among these, 2 clusters (OS-M1 and M2, 62.5% of 1,113 genes) showed progressive downregulation over time (FIGS. 3a, b). Among these downregulated genes, 70 genes encoded mitoribosomal proteins according to MitoCarta3.0 (FIG. 3c).

In the two analyzed cell senescence models (RS and OS), 51 MRPs (67.1% of a total of 82 MRPs) were generally downregulated at early time points (FIG. 3d), which highlighted the potential involvement of the senescence-associated (SA)-MRP gene signatures in OXPHOS dysfunction and the consequent deregulation of mitochondrial bioenergetics.

### Example 3

Downregulated Protein Expression of MRP which is Associated with Early Senescence Phenotype and Mitochondrial ROS Generation.

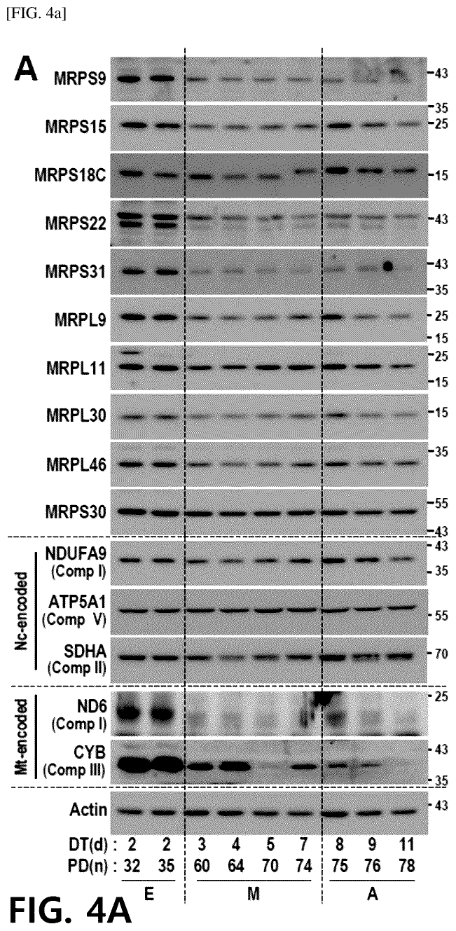

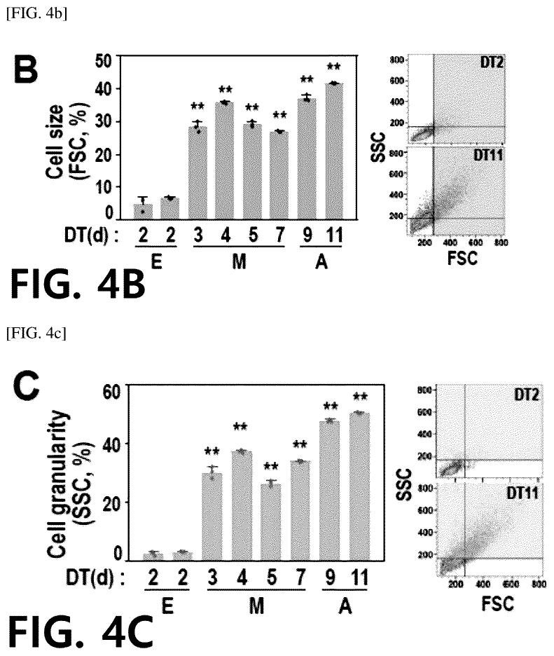

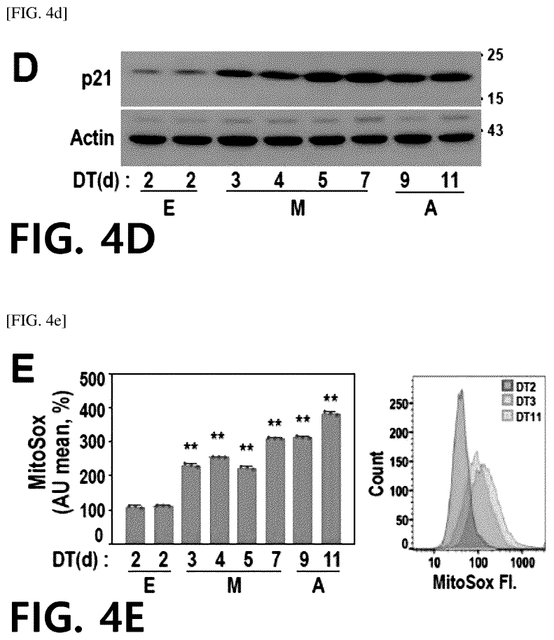

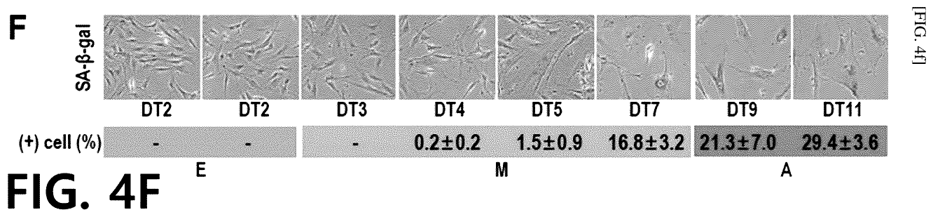

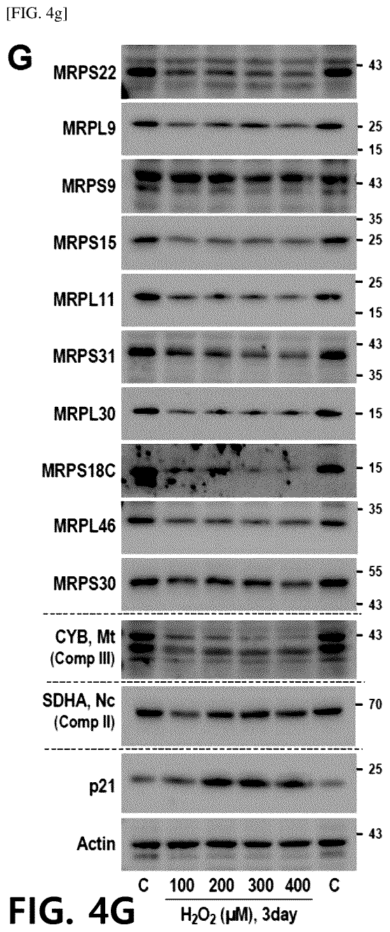

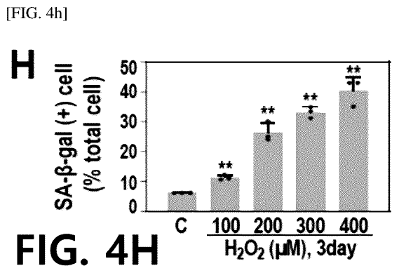

In order to estimate the functional expression of SA-MRP features, the inventors of the present invention next investigated the protein expressions of several SA-MRPs using the RS model of HDF. In particular, the protein levels of 10 selected SA-MRPs were reduced in M phase, with the inhibition of mtDNA-encoded OXPHOS proteins (ND6 and CYB) without alteration of ncDNA-encoded OXPHOS proteins (NDUFA9, ATP5A1 and SDHA) (FIG. 4a and FIG. 5). These results indicate that downregulated SA-MRP features may be closely associated with mitoribosome translation activity.

At the same time, the inventors of the present invention observed an increase in cell size and cell granularity as well as the induction of p21 (FIG. 4b-d), and this represents an early senescence phenotype acquired before the increased activity of senescence-associated galactosidase (SA-β-gal), which is a well-established senescence marker (FIG. 4f). Additionally, the level of mitochondrial reactive species (ROS) increased in the M phase (FIG. 4e), suggesting the dysfunction of mitochondrial OXPHOS. In accordance with the results observed in the RS model, the expression levels of SA-MRP protein and Medina encoding protein (CYB) were decreased in the OS model (FIGS. 4g, h and FIG. 6).

### Example 4

Mitoribosome Perturbation that Causes Cell Senescence

The inventors of the present invention sought to investigate whether functional defects in mitoribosome translation activity could cause cellular senescence.

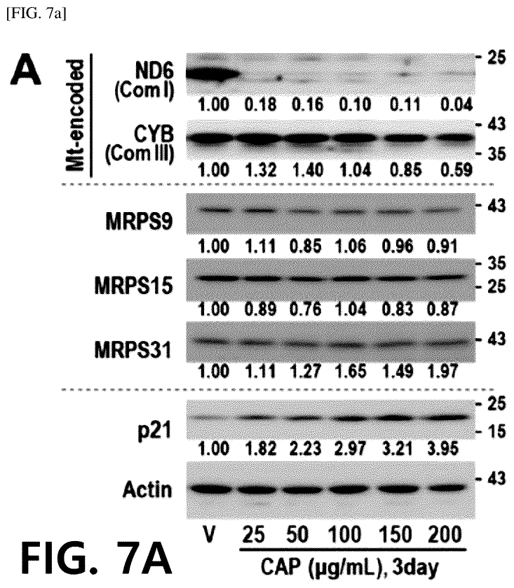

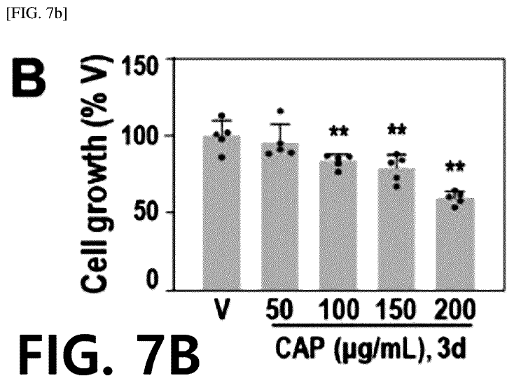

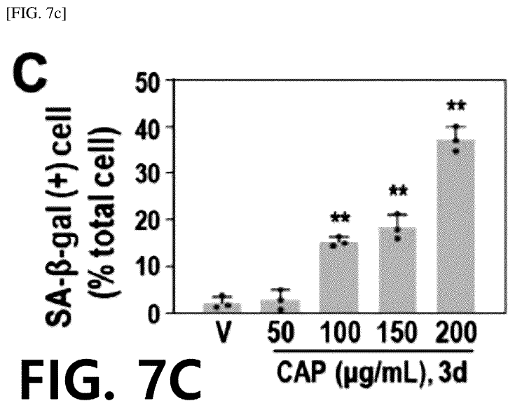

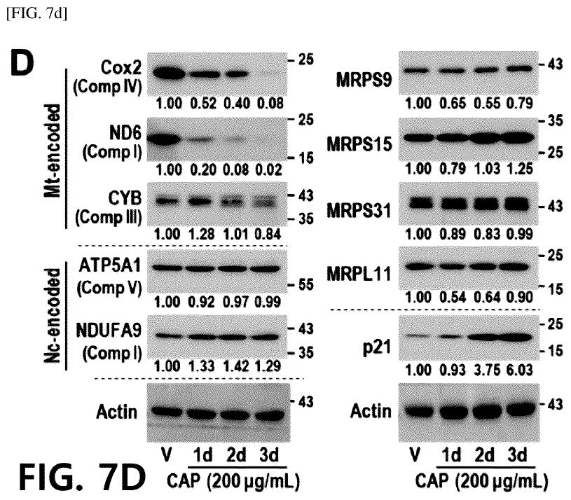

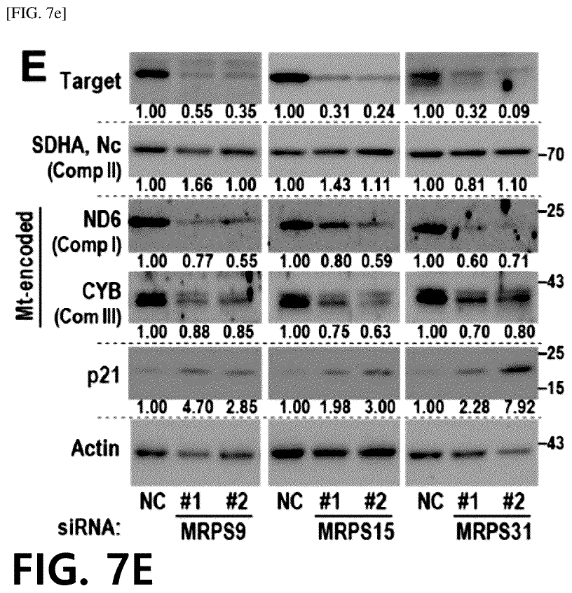

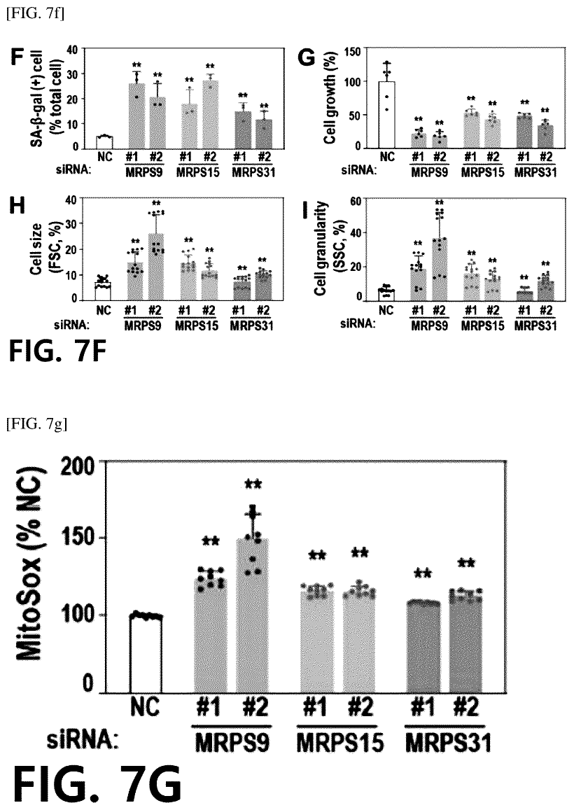

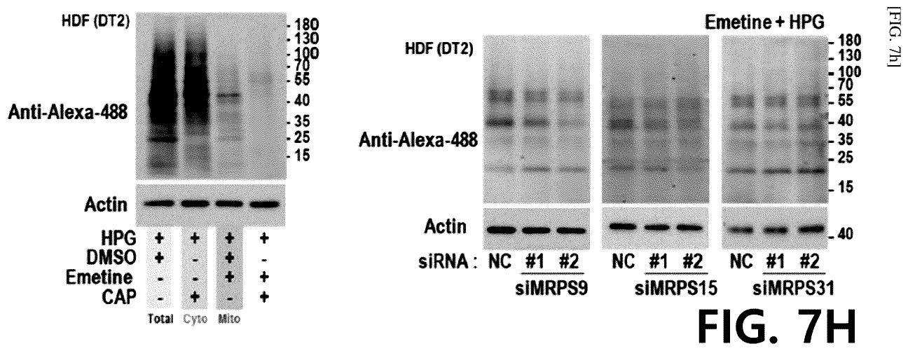

Young HDFs (DT2) exposed to chloramphenicol (CAP), which is a selective pharmacological inhibitor of mitochondrial translation, showed a significant decrease in the protein levels of ND6 and CYB, but not in the levels of MRP (FIG. 7a). Additionally, higher concentrations (>100 μg/mL) of CAP significantly induced a senescence phenotype with reduced cell growth, and increased SA-β-gal activity and p21 induction (FIGS. 7a-7c). Additionally, CAP-induced reduction in the protein expression of mtDNA-encoded proteins (COX2, ND, and CYB) and induction of p21 were observed over time (FIG. 7d).

In order to investigate the genetic repression effect of SA-MRP, the inventors of the present invention targeted 3 small mitoribosome subunit proteins, MRPS9, MRPS15 and MRPS31, which can effectively block the initiation step of mitochondrial translation. siRNA-mediated knockdown of the 3 types of MRPs effectively suppressed the protein expressions of ND6 and CYB (FIG. 7e) and significantly induced senescence as evidenced by an increase in SA-β-gal-positive cells, cell size and particle size, and a decrease in cell growth (FIG. 7f). Importantly, mitochondrial ROS increased (FIG. 7g), and mitochondrial translation activity decreased (FIG. 7h). These data indicate that the deregulation of mitoribosomes causes mitochondrial dysfunction and induces cell senescence.

### Example 5

Reduced Protein Expression of TPP1 which is an Early Event Leading to Cellular Senescence.

In order to elucidate the link between mitoribosome deregulation and telomere damage, the inventors of the present invention monitored telomere length in cells during the E and M phases of RS.

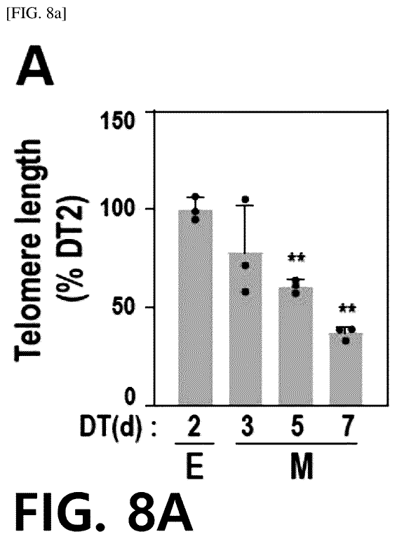

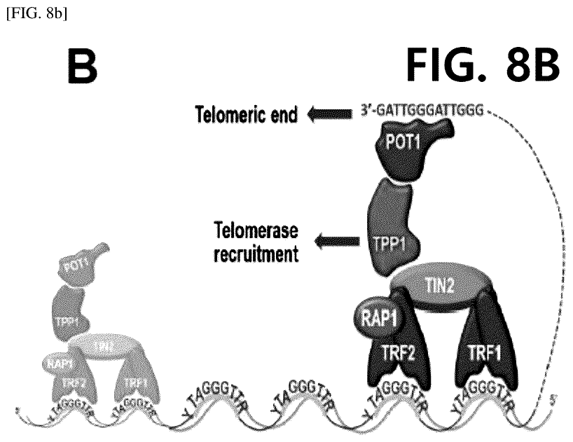

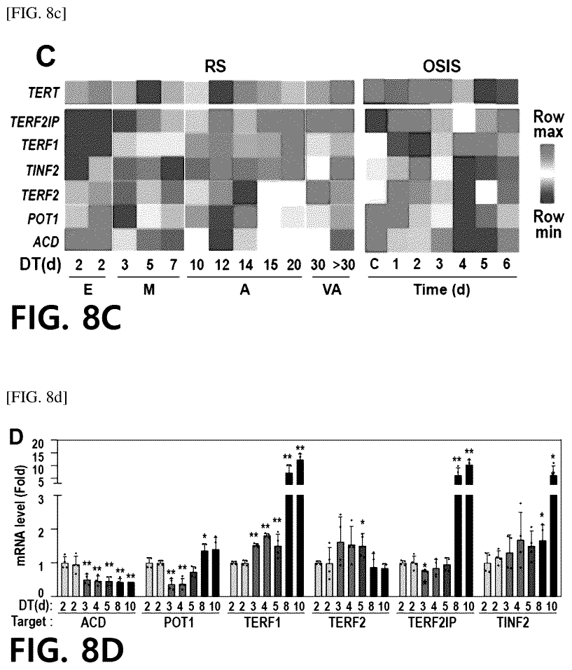

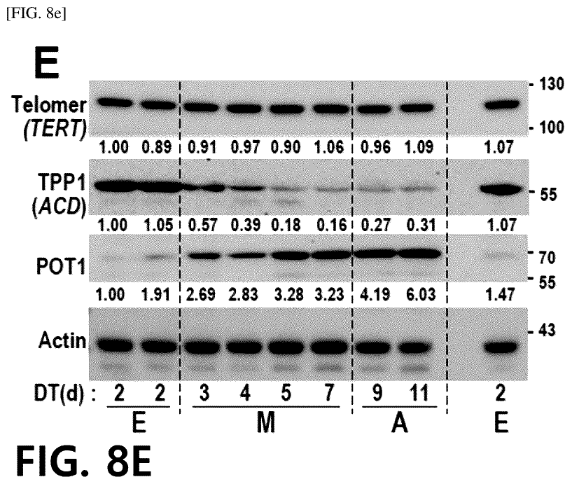

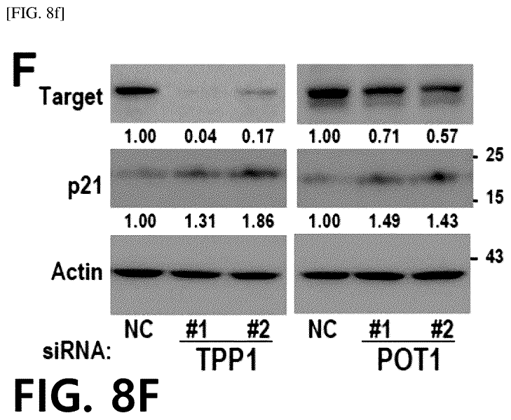

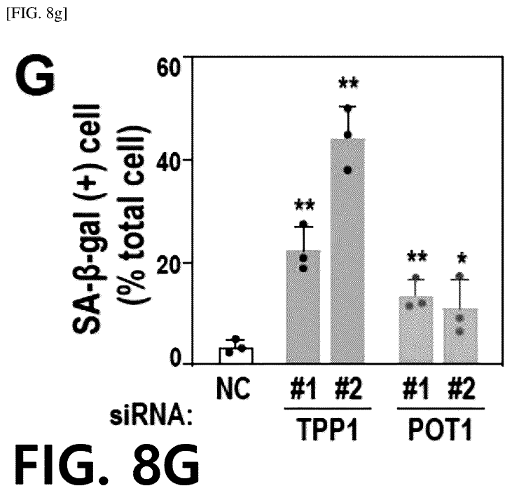

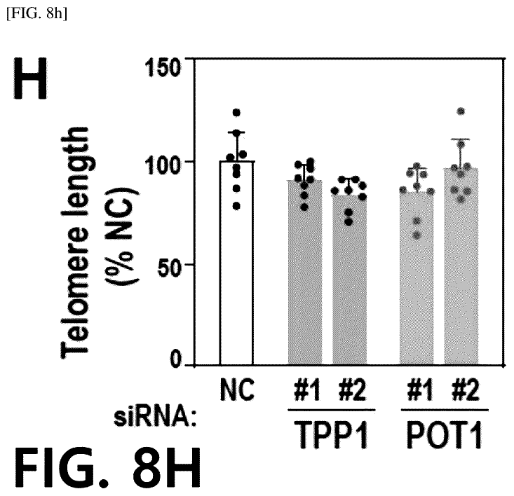

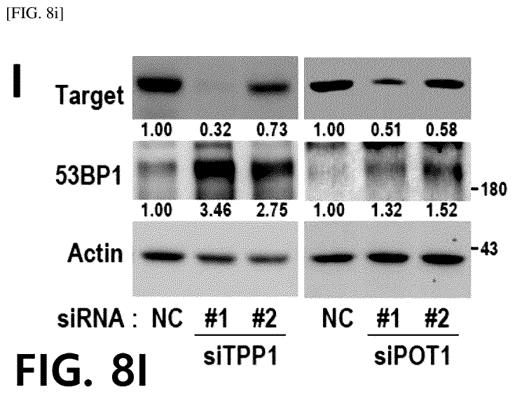

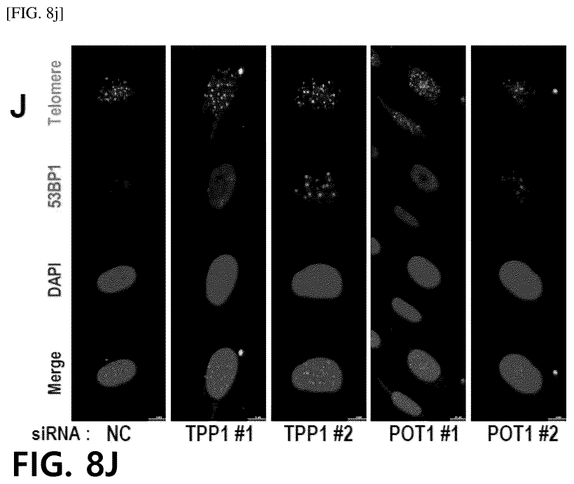

Compared to cells in the E phase (DT2), cells in the M phase presented shorter telomere lengths (FIG. 8a). Telomere ends are protected by a shelterin complex, which consists of six proteins including TRF1, TRF2, RAP1, POT1, TIN2 and TPP1 (FIG. 8b), and the length is maintained by telomerase activity, which is regulated by the complex depending on the cell type (cancer cells and normal cells). Expression profiles of the six shelterin genes derived from the RS transcript showed a significant decrease in the expression of ACD (named TPP1 for the protein) with a slight decrease in the expression of POT1 after the M stage (FIG. 8c, left and FIG. 8d). In the OS model, ACD expression decreased in H₂O₂-treated HDFs from day 1 (FIG. 8c, right). However, at the protein level, only TPP1, but not POT1, was decreased in the M phase of the RS model (FIG. 8e), indicating the functional involvement of TPP1 in the initial entry into RS. The knockdown of TPP1 significantly induced a senescence phenotype, including increased SA-β-gal activity and induction of p21, whereas POT1 knockdown induced senescence to a lesser extent (FIGS. 8f, g). However, the genetic inhibition of shelterin protein did not substantially affect telomere length (FIG. 8h). These results suggested that functional defects in the shelterin complex can induce senescence without telomere attrition, regardless of genetic origin.

Next, the inventors of the present invention further investigated how TPP1 inhibition induces senescence without telomere attrition. Recently, the involvement of telomeric DNA damage in cellular senescence has been demonstrated. In order to monitor DNA damage in telomeric regions, the inventors of the present invention examined the expression of 53BP1, which is a key player in the DNA damage response, and its localization to telomeric DNA, known as telomere dysfunction-inducing foci (TIFs). The inhibition of TPP1 induced increased cellular 53BP1 expression by forming punctate foci within the nucleus (FIGS. 8i, j) and increased TIF, of which 53BP1 foci were closely localized to telomeric regions (FIGS. 8j and 9A), indicating DNA damage at telomere ends.

Telomere dysfunction-induced foci (TIFs), colocalized foci of telomeres (green) and 53BP1 (red) were visualized by IF-FISH as described in Materials and Methods. Nuclei were visualized using DAPI (blue) staining. The quantification of TIF is shown in FIG. 9A.

### Example 6

### Upstream Mitoribosome Deregulation Function of Transcriptional TPP1 Inhibition

The inventors of the present invention investigated the interrelationship between mitoribosome deregulation and TPP1 inhibition.

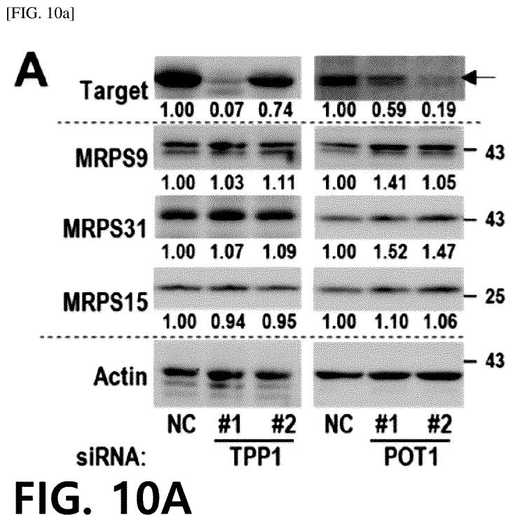

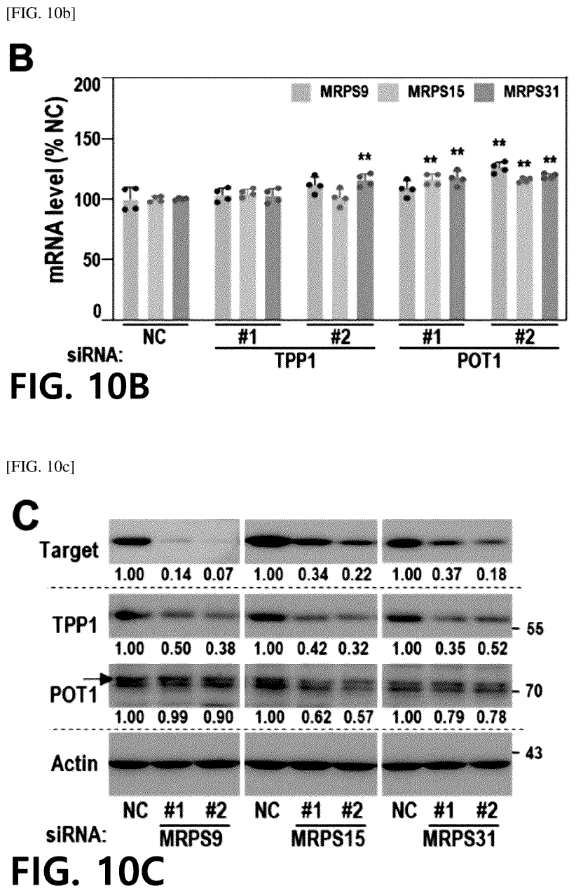

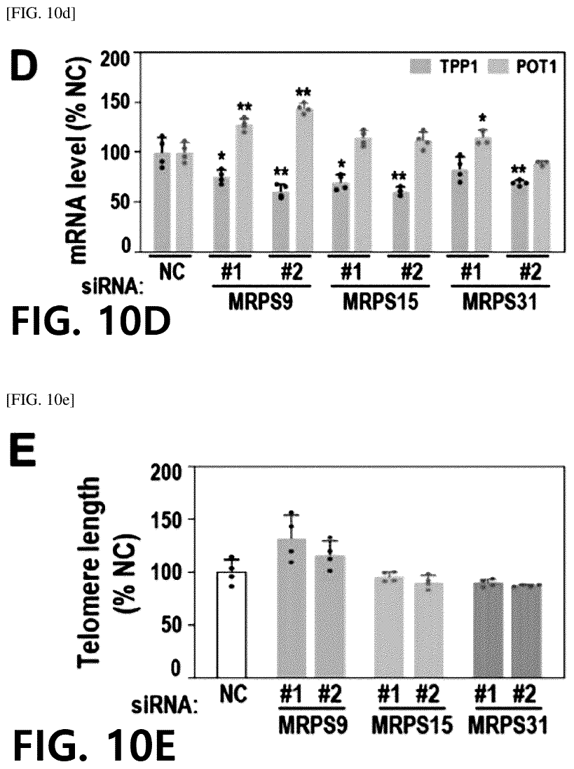

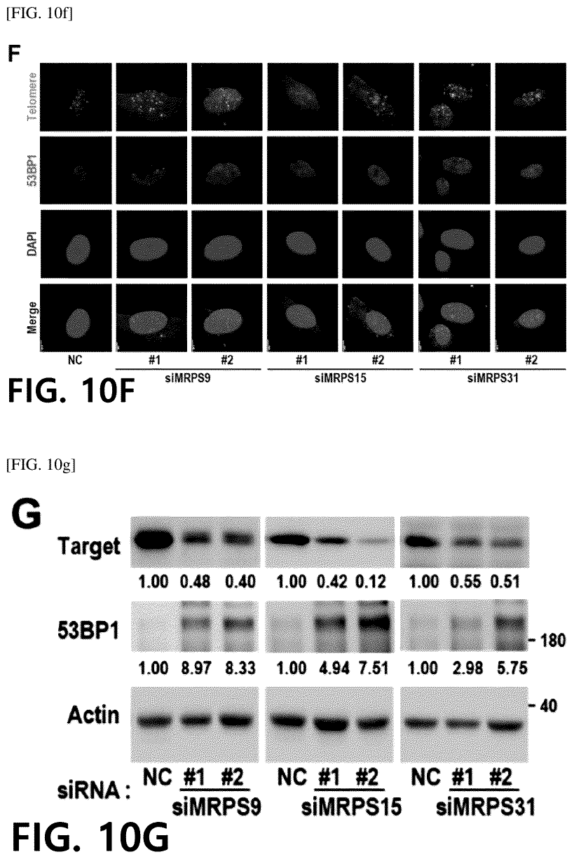

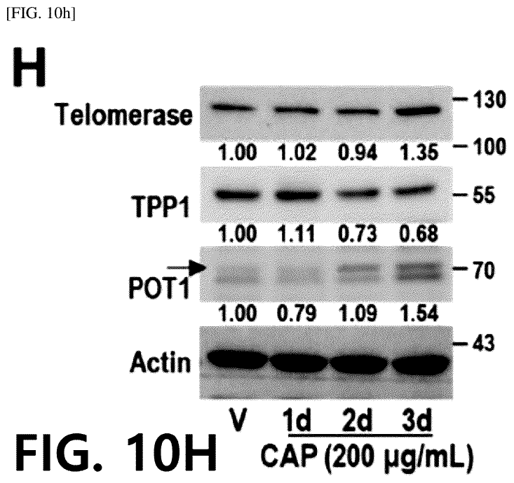

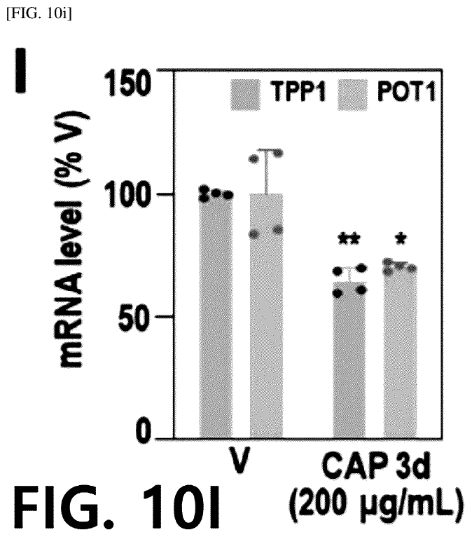

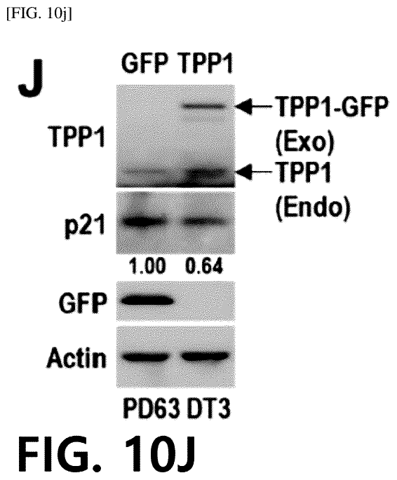

Knockdown of TPP1 or POT1 did not affect the mRNA and protein expression of three MRPs (MRPS9, MRPS31, and MRPS15) (FIGS. 10a, b). However, individual knockdown of the three MRPs reduced POT1 expression and selectively reduced mRNA and protein expression of TPP1 without altering telomere length (FIG. 10c-e). Increased 53BP1 expression and TIF were also observed upon knockdown of the three MRPs (FIGS. 10f, g and FIG. 9B). Additionally, CAP-mediated inhibition of mitoribosome activity decreased the expression level of TPP1, but not POT1 and telomerase (FIGS. 10h, i). When TPP1 was overexpressed in M-stage HDFs (PD63, DT3), p21 induction was reduced, indicating a delayed senescence process (FIG. 10j). These findings indicate that the deregulation of mitoribosomes serves as an upstream regulator of TPP1 inhibition-mediated senescence.

### Example 7

### Gene Expression of MRP and Shelterin Complex Downregulated in Skin Tissue

In order to verify the deregulation of MRP and shelter complex genes in senescent cells and tissues, the inventors of the present invention next analyzed publicly available transcriptomes of skin tissues and isolated primary cells.

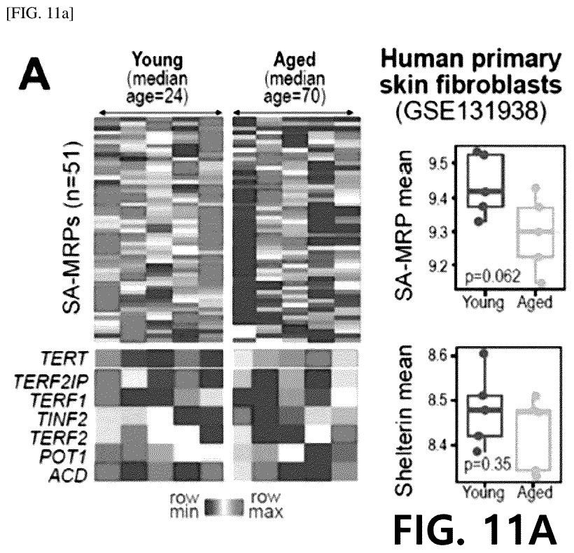

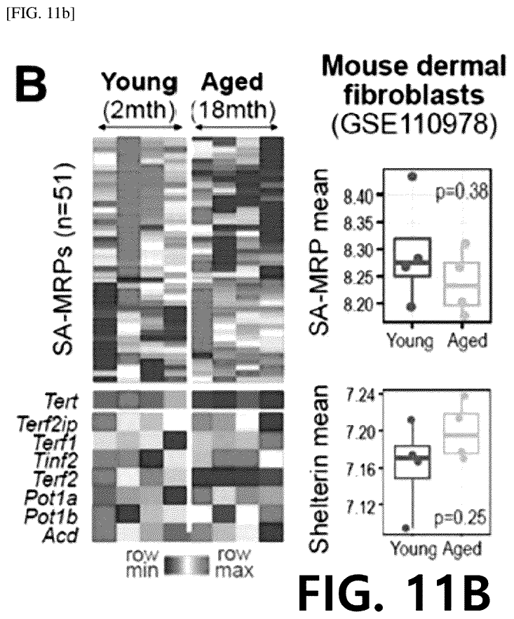

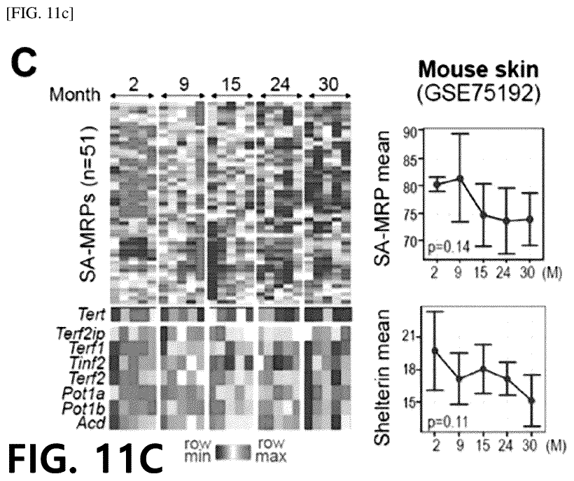

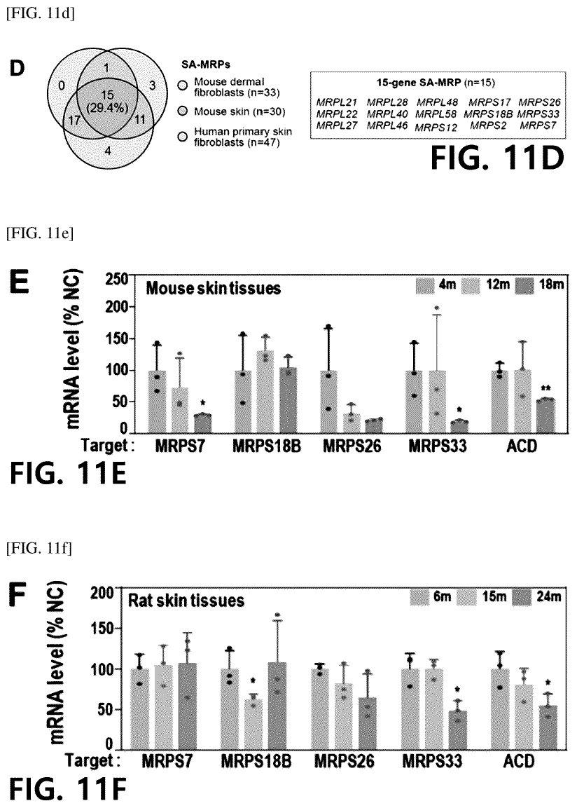

Compared to human primary skin fibroblasts from young donors, skin fibroblasts from older donors had reduced expression levels of 51 SA-MRPs, but this did not reach statistical significance (p=0.062, FIG. 11a). Similar results were obtained in mouse primary dermal fibroblasts and skin tissue (FIGS. 11b, c). Using the expression profiles of 51 SA-MRPs, unsupervised hierarchical clustering of the two analyzed data sets identified specific SA-MRPs that were significantly downregulated in senescent cells and skin tissues (FIG. 5), and among human and mouse fibroblasts, 15 SA-MRPs were generally downregulated (FIG. 11d). However, slightly reduced expression of ACD was observed only in senescent human cells (FIG. 11a-c), while the expression of the shelterin gene did not reach statistical significance. TERT was slightly increased in aged human cells but decreased in aged mouse fibroblasts and tissues (FIG. 11a-c), indicating differential involvement of telomerase in the two species. In particular, the expression levels of SA-MRP and shelterin genes in mouse skin tissue gradually decreased with age (FIG. 11c).

In order to verify the above results, the inventors of the present invention performed qRT-PCR using mouse skin tissues obtained at three different ages (4, 12 and 18 months), and in three samples, the inventors of the present invention discovered that the mRNA expression levels of specific SA-MRPs, including Mrps7, Mrps33, Mrps26 and Acd, were decreased (FIG. 11e). Similarly, reduced expression levels of MRPS33 and Acd were further confirmed using aged mouse skin tissue (6, 15 and 24 months) (FIG. 11f).

### Example 8

### Single Cell Transcriptome of Human Skin Showing Early Suppressed Expression of SA-MRP and Shelterin Genes.

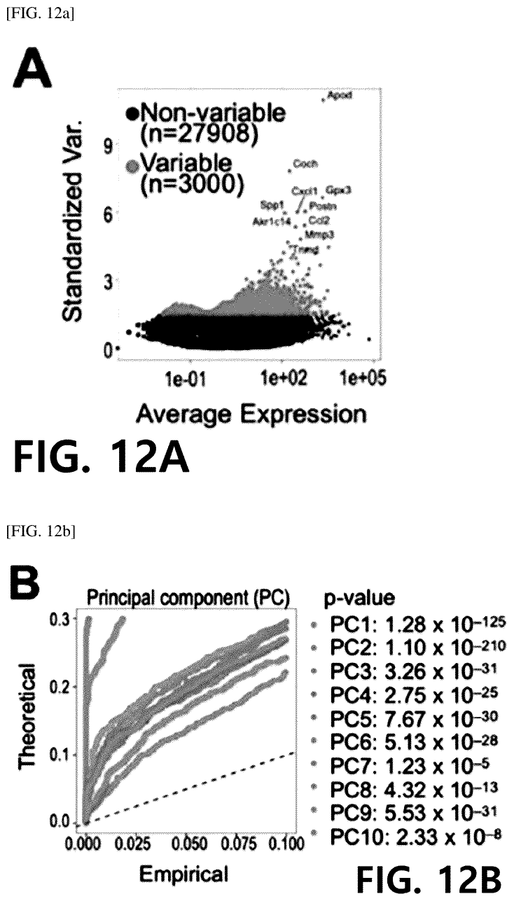

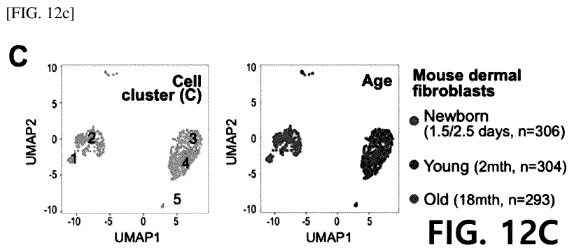

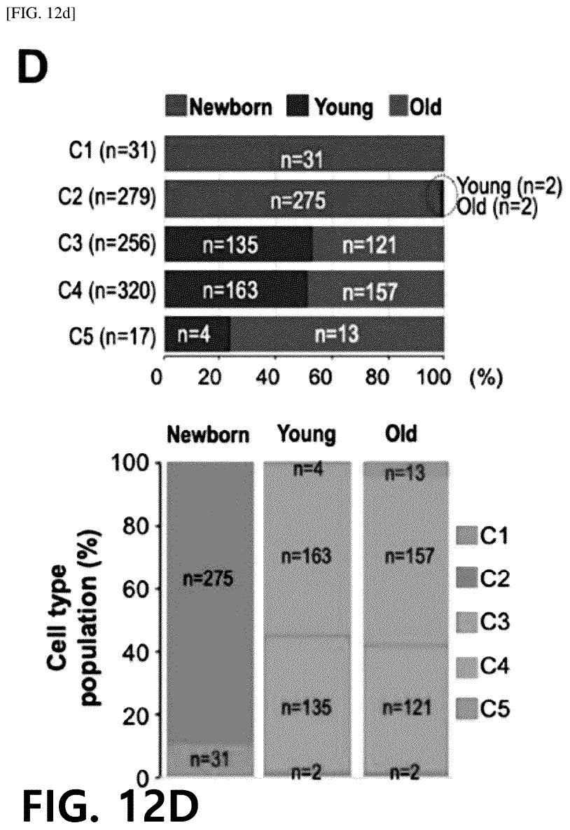

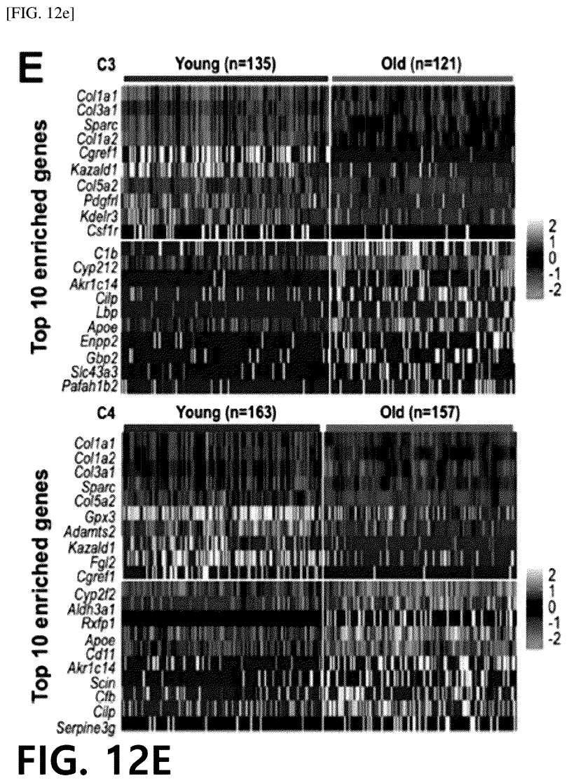

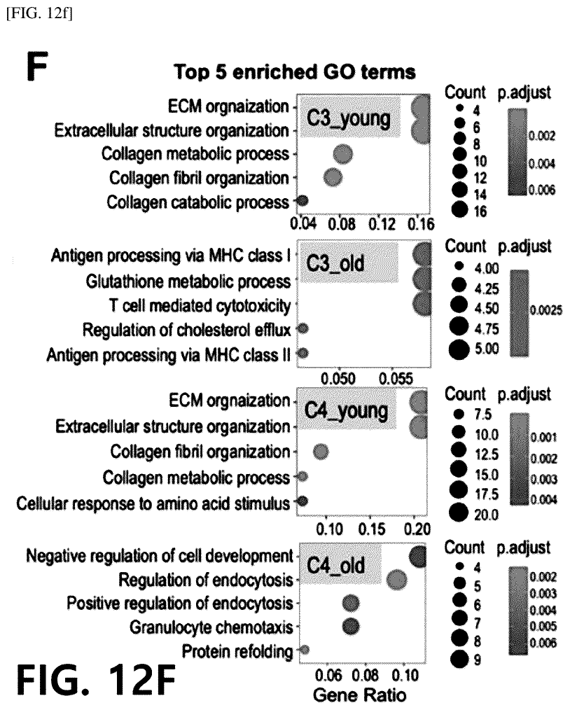

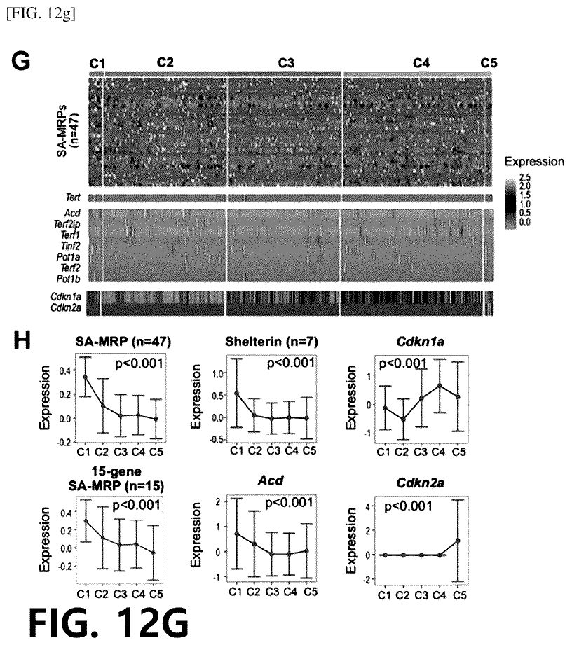

In order to assess the cellular heterogeneity of SA-MRP and shelter complex expressions and their association, the inventors of the present invention processed and analyzed scRNA-seq data sets derived from senescence skin fibroblasts. Principal component analysis (PCA) was performed on the expanded data consisting of 1,044 QC-passed cells and 3,000 highly variable features to determine the number of principal components (PCs) for cell clustering (FIGS. 12a, b). The first 10 PCs were used to cluster senescent skin fibroblasts of mice from three different ages (newborn (P1.5-2.5 days), young (2 months) and old (18 months)) into six cell subpopulations. One cluster of cells (C6) predominantly expressing mitochondrial genes was excluded from further analysis (FIG. 6). Among the remaining five cell clusters (labeled C1-C5), the UMAP plot clearly distinguishes that cells in C1 and C2 are mostly derived from fibroblasts from newborn mice, while cells in C3-C5 are derived from young and old mice. (FIG. 12c).

Identified cell subpopulations included proportions of cells of varying ages, suggesting heterogeneous age-defining characteristics of cell clusters (FIG. 12d). The inventors of the present invention also discovered that the cell subpopulations of fibroblasts in young mice were similar to those in old mice. Gene ontology (GO) analysis of the highest enriched functions in C3 and C4 cells identified extracellular matrix (ECM) tissue and extracellular structural tissue as the highest enriched GO terms in young mice, which was consistent with recent scRNA-seq findings on human skin from young donors (FIGS. 12e, f and FIG. 9). Although the PCA data suggest that skin tissue from young and old mice has a similar composition in terms of cell subpopulations, downstream analysis clearly indicates that different cell subpopulations uniquely express age-defining functions, thereby highlighting the importance of cluster determination functions present in young and aged mice and deciphering fibroblast heterogeneity in senescent skin. Importantly, the inventors of the present invention discovered that the expression of MRP and shelterin complex components, including Acd, gradually decreased in cells from C3 to C5 compared to C1 and C2, as the expression of p21 and p16 increased (FIG. 12g). These results suggest that the deregulation of SA-MRP and Acd occurs before mice reach 2 months of age, supporting the argument of the inventors of the present invention for a potential role for SA-MRP and TPP1 as regulators of the early stages of senescence.

### INDUSTRIAL APPLICABILITY

Since the expression level of the mitoribosomal protein of the present invention decrease in the early stages of senescence, it can be effectively utilized in a biomarker composition for diagnosing senescence, a kit for diagnosing senescence including the same, or a method for providing information for diagnosing senescence, and thus, it has industrial applicability.

### SEQUENCE LISTING FREE TEXT

SEQ ID NO: 1 represents a #1 sequence of target siRNA duplexes for MRPS9.

SEQ ID NO: 2 represents a #2 sequence of target siRNA duplexes for MRPS9.

SEQ ID NO: 3 represents a #1 sequence of target siRNA duplexes for MRPS15.

SEQ ID NO: 4 represents a #2 sequence of target siRNA duplexes for MRPS15.

SEQ ID NO: 5 represents a #1 sequence of target siRNA duplexes for MRPS31.

SEQ ID NO: 6 represents a #2 sequence of target siRNA duplexes for MRPS31.

SEQ ID NO: 7 represents a #1 sequence of target siRNA duplexes for ACD.

SEQ ID NO: 8 represents a #2 sequence of target siRNA duplexes for ACD.

SEQ ID NO: 9 represents a #1 sequence of target siRNA duplexes for POT1.

SEQ ID NO: 10 represents a #2 sequence of target siRNA duplexes for POT1.

SEQ ID NO: 11 represents a sequence of target siRNA duplexes for the negative control group.

SEQ ID NO: 12 represents a forward primer sequence of Human MRPS9.

SEQ ID NO: 13 represents a reverse primer sequence of Human MRPS9.

SEQ ID NO: 14 represents a forward primer sequence of Human MRPS15.

SEQ ID NO: 15 represents a reverse primer sequence of Human MRPS15.

SEQ ID NO: 16 represents a forward primer sequence of Human MRPS31.

SEQ ID NO: 17 represents a reverse primer sequence of Human MRPS31.

SEQ ID NO: 18 represents a forward primer sequence of Human ACD.

SEQ ID NO: 19 represents a reverse primer sequence of Human ACD.

SEQ ID NO: 20 represents a forward primer sequence of Human POT1.

SEQ ID NO: 21 represents a reverse primer sequence of Human POT1.

SEQ ID NO: 22 represents a forward primer sequence of Human TERF1.

SEQ ID NO: 23 represents a reverse primer sequence of Human TERF1.

SEQ ID NO: 24 represents a forward primer sequence of Human TINF2.

SEQ ID NO: 25 represents a reverse primer sequence of Human TINF2.

SEQ ID NO: 26 represents a forward primer sequence of Human TERF2.

SEQ ID NO: 27 represents a reverse primer sequence of Human TERF2.

SEQ ID NO: 28 represents a forward primer sequence of Human TERF2IP.

SEQ ID NO: 29 represents a reverse primer sequence of Human TERF2IP.

SEQ ID NO: 30 represents a forward primer sequence of Human TBP.

SEQ ID NO: 31 represents a reverse primer sequence of Human TBP.

SEQ ID NO: 32 represents a forward primer sequence of Mouse Mrps7.

SEQ ID NO: 33 represents a reverse primer sequence of Mouse Mrps7.

SEQ ID NO: 34 represents a forward primer sequence of Mouse Mrps18b.

SEQ ID NO: 35 represents a reverse primer sequence of Mouse Mrps18b.

SEQ ID NO: 36 represents a forward primer sequence of Mouse Mrps26.

SEQ ID NO: 37 represents a reverse primer sequence of Mouse Mrps26.

SEQ ID NO: 38 represents a forward primer sequence of Mouse Mrps33.

SEQ ID NO: 39 represents a reverse primer sequence of Mouse Mrps33.

SEQ ID NO: 40 represents a forward primer sequence of Mouse Acd.

SEQ ID NO: 41 represents a reverse primer sequence of Mouse Acd.

SEQ ID NO: 42 represents a forward primer sequence of Mouse Tbp.

SEQ ID NO: 43 represents a reverse primer sequence of Mouse Tbp.

SEQ ID NO: 44 represents a forward primer sequence of Rat Mrps7.

SEQ ID NO: 45 represents a reverse primer sequence of Rat Mrps7.

SEQ ID NO: 46 represents a forward primer sequence of Rat Mrps18b.

SEQ ID NO: 47 represents a reverse primer sequence of Rat Mrps18b.

SEQ ID NO: 48 represents a forward primer sequence of Rat Mrps26.

SEQ ID NO: 49 represents a reverse primer sequence of Rat Mrps26.

SEQ ID NO: 50 represents a forward primer sequence of Rat Mrps33.

SEQ ID NO: 51 represents a reverse primer sequence of Rat Mrps33.

SEQ ID NO: 52 represents a forward primer sequence of Rat Acd.

SEQ ID NO: 53 represents a reverse primer sequence of Rat Acd.

SEQ ID NO: 54 represents a forward primer sequence of Rat Tbp.

SEQ ID NO: 55 represents a reverse primer sequence of Rat Tbp.

SEQ ID NO: 56 represents a primer sequence for telomeres.

SEQ ID NO: 57 represents a primer sequence for telomeres.

SEQ ID NO: 58 represents a primer sequence for 36B4.

SEQ ID NO: 59 represents a primer sequence for 36B4.

1. A biomarker composition for diagnosing senescence, comprising an agent for detecting any one or more mitoribosomal proteins selected from the group consisting of MRPL21, MRPL28, MRPS17, MRPL40, MRPL58, MRPS12, MRPS2 and MRPS7, or a gene encoding the mitoribosomal protein.

2. The biomarker composition of claim 1, wherein the senescence is in early stages of senescence.

3. The biomarker composition of claim 2, wherein the early stages of senescence are before activation of senescence β-galactosidase (SA-β-gal).

4. The biomarker composition of claim 1, wherein the senescence is skin senescence.

5. A kit for diagnosing senescence, comprising the biomarker composition of claim 1.

6. A method for diagnosing senescence, the method comprising:
i) measuring an expression level of any one or more mitoribosomal proteins selected from the group consisting of MRPL21, MRPL28, MRPS17, MRPL40, MRPL58, MRPS12, MRPS2 and MRPS7, or a gene encoding the mitoribosomal protein in a sample isolated from a subject;
ii) comparing the measured expression level with a control sample; and
iii) classifying the measured expression level as senescence if the measured expression level is lower than an expression level of the control sample.

7. The method of claim 6, wherein the senescence is in early stages of senescence.

8. The method of claim 7, wherein the early stages of senescence are before activation of senescence β-galactosidase (SA-β-gal).

9. The method of claim 6, wherein the senescence is skin senescence.


SEQUENCE LISTING (59 sequences)

SEQ ID NO: 1 - RNA, 19 nt, synthetic construct
  misc_feature 1..19: note = siRNA duplexes_MRPS9_#1
  source 1..19: mol_type = other RNA
gaggtttcgaagaagtgta

SEQ ID NO: 2 - RNA, 20 nt, synthetic construct
  misc_feature 1..20: note = siRNA duplexes_MRPS9_#2
  source 1..20: mol_type = other RNA
gaaaacttacacagaggatt

SEQ ID NO: 3 - RNA, 19 nt, synthetic construct
  misc_feature 1..19: note = siRNA duplexes_MRPS15_#1
  source 1..19: mol_type = other RNA
cacttggagaaacatcgaa

SEQ ID NO: 4 - RNA, 19 nt, synthetic construct
  misc_feature 1..19: note = siRNA duplexes_MRPS15_#2
  source 1..19: mol_type = other RNA
gtctgtcaagatccgcagt

SEQ ID NO: 5 - RNA, 19 nt, synthetic construct
  misc_feature 1..19: note = siRNA duplexes_MRPS31_#1
  source 1..19: mol_type = other RNA
ctcggcgatgtttcctaga

SEQ ID NO: 6 - RNA, 19 nt, synthetic construct
  misc_feature 1..19: note = siRNA duplexes_MRPS31_#2
  source 1..19: mol_type = other RNA
gattctctcccttttgata

SEQ ID NO: 7 - RNA, 19 nt, synthetic construct
  misc_feature 1..19: note = siRNA duplexes_ACD_#1
  source 1..19: mol_type = other RNA
gacctatctctgaccctca

SEQ ID NO: 8 - RNA, 19 nt, synthetic construct
  misc_feature 1..19: note = siRNA duplexes_ACD_#2
  source 1..19: mol_type = other RNA
cctaccggtaccacaggaa

SEQ ID NO: 9 - RNA, 19 nt, synthetic construct
  misc_feature 1..19: note = siRNA duplexes_POT1_#1
  source 1..19: mol_type = other RNA
ctgatgtggatcaactgaa

SEQ ID NO: 10 - RNA, 19 nt, synthetic construct
  misc_feature 1..19: note = siRNA duplexes_POT1_#2
  source 1..19: mol_type = other RNA
ctgatcaagctacagtgta

SEQ ID NO: 11 - RNA, 19 nt, synthetic construct
  misc_feature 1..19: note = siRNA duplexes_negative control
  source 1..19: mol_type = other RNA
cctacgccaccaatttggt

SEQ ID NO: 12 - DNA, 21 bp, synthetic construct
  misc_feature 1..21: note = MRPS9_primer_F
  source 1..21: mol_type = other DNA
atcacacaggacagagaacag

SEQ ID NO: 13 - DNA, 20 bp, synthetic construct
  misc_feature 1..20: note = MRPS9_primer_R
  source 1..20: mol_type = other DNA
attgccagtcgtattgctcc

SEQ ID NO: 14 - DNA, 21 bp, synthetic construct
  misc_feature 1..21: note = MRPS15_primer_F
  source 1..21: mol_type = other DNA
agcgccaagtttcctttcaac

SEQ ID NO: 15 - DNA, 20 bp, synthetic construct
  misc_feature 1..20: note = MRPS15_primer_R
  source 1..20: mol_type = other DNA
ctttgagcagcgtagaggga

SEQ ID NO: 16 - DNA, 20 bp, synthetic construct
  misc_feature 1..20: note = MRPS31_primer_F
  source 1..20: mol_type = other DNA
gacgttcctacctcttcgcc

SEQ ID NO: 17 - DNA, 20 bp, synthetic construct
  misc_feature 1..20: note = MRPS31_primer_R
  source 1..20: mol_type = other DNA
ctgcggtacctgactgttcc

SEQ ID NO: 18 - DNA, 21 bp, synthetic construct
  misc_feature 1..21: note = ACD_primer_F
  source 1..21: mol_type = other DNA
ttgaggtactacaggacgccg

SEQ ID NO: 19 - DNA, 21 bp, synthetic construct
  misc_feature 1..21: note = ACD_primer_R
  source 1..21: mol_type = other DNA
cgaactccttctcctcccagt

SEQ ID NO: 20 - DNA, 20 bp, synthetic construct
  misc_feature 1..20: note = POT1_primer_F
  source 1..20: mol_type = other DNA
gccttacgtgtttgggcatc

SEQ ID NO: 21 - DNA, 20 bp, synthetic construct
  misc_feature 1..20: note = POT1_primer_R
  source 1..20: mol_type = other DNA
tggtgccatcccataccttt

SEQ ID NO: 22 - DNA, 20 bp, synthetic construct
  misc_feature 1..20: note = TERF1_primer_F
  source 1..20: mol_type = other DNA
cagcgcagaggctattattc

SEQ ID NO: 23 - DNA, 19 bp, synthetic construct
  misc_feature 1..19: note = TERF1_primer_R
  source 1..19: mol_type = other DNA
tcaaactgtgcatcaaggg

SEQ ID NO: 24 - DNA, 20 bp, synthetic construct
  misc_feature 1..20: note = TINF2_primer_F
  source 1..20: mol_type = other DNA
cagggggaaggaaaggaatc

SEQ ID NO: 25 - DNA, 20 bp, synthetic construct
  misc_feature 1..20: note = TINF2_primer_R
  source 1..20: mol_type = other DNA
gaggcaggagactagagtac

SEQ ID NO: 26 - DNA, 20 bp, synthetic construct
  misc_feature 1..20: note = TERF2_primer_F
  source 1..20: mol_type = other DNA
tcgagaaaagaacttggccc

SEQ ID NO: 27 - DNA, 20 bp, synthetic construct
  misc_feature 1..20: note = TERF2_primer_R
  source 1..20: mol_type = other DNA
cctttttggccatcgtgagg

SEQ ID NO: 28 - DNA, 20 bp, synthetic construct
  misc_feature 1..20: note = TERF2IP_primer_F
  source 1..20: mol_type = other DNA
gacgttcctacctcttcgcc

SEQ ID NO: 29 - DNA, 20 bp, synthetic construct
  misc_feature 1..20: note = TERF2IP_primer_R
  source 1..20: mol_type = other DNA
ctgcggtacctgactgttcc

SEQ ID NO: 30 - DNA, 18 bp, synthetic construct
  misc_feature 1..18: note = TBP_primer_F
  source 1..18: mol_type = other DNA
cggatagcggggaaccac

SEQ ID NO: 31 - DNA, 21 bp, synthetic construct
  misc_feature 1..21: note = TBP_primer_R
  source 1..21: mol_type = other DNA
cacaagcatctttttgactgc

SEQ ID NO: 32 - DNA, 19 bp, synthetic construct
  misc_feature 1..19: note = Mrps7_primer_F
  source 1..19: mol_type = other DNA
aaccttccagggctcacac

SEQ ID NO: 33 - DNA, 20 bp, synthetic construct
  misc_feature 1..20: note = Mrps7_primer_R
  source 1..20: mol_type = other DNA
gctcctggtcatacttctcc

SEQ ID NO: 34 - DNA, 19 bp, synthetic construct
  misc_feature 1..19: note = Mrps18b_primer_F
  source 1..19: mol_type = other DNA
cgcttctaaagctggttcc

SEQ ID NO: 35 - DNA, 19 bp, synthetic construct
  misc_feature 1..19: note = Mrps18b_primer_R
  source 1..19: mol_type = other DNA
agcggttgtggtattcttc

SEQ ID NO: 36 - DNA, 19 bp, synthetic construct
  misc_feature 1..19: note = Mrps26_primer_F
  source 1..19: mol_type = other DNA
cctgccaaatccaaggtcg

SEQ ID NO: 37 - DNA, 20 bp, synthetic construct
  misc_feature 1..20: note = Mrps26_primer_R
  source 1..20: mol_type = other DNA
ccaacgtgaactctagcctg

SEQ ID NO: 38 - DNA, 20 bp, synthetic construct
  misc_feature 1..20: note = Mrps33_primer_F
  source 1..20: mol_type = other DNA
cgaggtcaggagcaggaatc

SEQ ID NO: 39 - DNA, 19 bp, synthetic construct
  misc_feature 1..19: note = Mrps33_primer_R
  source 1..19: mol_type = other DNA
aaacaggctcaccactttc

SEQ ID NO: 40 - DNA, 17 bp, synthetic construct
  misc_feature 1..17: note = Acd_primer_F
  source 1..17: mol_type = other DNA
tccaaactgccaggctc

SEQ ID NO: 41 - DNA, 17 bp, synthetic construct
  misc_feature 1..17: note = Acd_primer_R
  source 1..17: mol_type = other DNA
tcagcccatgtgtcctc

SEQ ID NO: 42 - DNA, 19 bp, synthetic construct
  misc_feature 1..19: note = Tbp_primer_F
  source 1..19: mol_type = other DNA
gtggatcgagtccggtagc

SEQ ID NO: 43 - DNA, 19 bp, synthetic construct
  misc_feature 1..19: note = Tbp_primer_R
  source 1..19: mol_type = other DNA
tgaaatagtgatgctgggc

SEQ ID NO: 44 - DNA, 21 bp, synthetic construct
  misc_feature 1..21: note = Mrps7_primer_F
  source 1..21: mol_type = other DNA
gaactcaccgaagaggagaag

SEQ ID NO: 45 - DNA, 23 bp, synthetic construct
  misc_feature 1..23: note = Mrps7_primer_R
  source 1..23: mol_type = other DNA
atgttggtgaatttgctgatgac

SEQ ID NO: 46 - DNA, 22 bp, synthetic construct
  misc_feature 1..22: note = Mrps18b_primer_F
  source 1..22: mol_type = other DNA
cgaaagacatgcattcgtaagg

SEQ ID NO: 47 - DNA, 20 bp, synthetic construct
  misc_feature 1..20: note = Mrps18b_primer_R
  source 1..20: mol_type = other DNA
ggcatggaagatgatacccg

SEQ ID NO: 48 - DNA, 20 bp, synthetic construct
  misc_feature 1..20: note = Mrps26_primer_F
  source 1..20: mol_type = other DNA
actgcaggaactacggatag

SEQ ID NO: 49 - DNA, 20 bp, synthetic construct
  misc_feature 1..20: note = Mrps26_primer_R
  source 1..20: mol_type = other DNA
tctttcagttgcacccaagc

SEQ ID NO: 50 - DNA, 20 bp, synthetic construct
  misc_feature 1..20: note = Mrps33_primer_F
  source 1..20: mol_type = other DNA
gagtgcccgtatctttggtg

SEQ ID NO: 51 - DNA, 22 bp, synthetic construct
  misc_feature 1..22: note = Mrps33_primer_R
  source 1..22: mol_type = other DNA
gtcctgatgctcatctctgtaa

SEQ ID NO: 52 - DNA, 19 bp, synthetic construct
  misc_feature 1..19: note = Acd_primer_F
  source 1..19: mol_type = other DNA
cccactctgtctgggaacc

SEQ ID NO: 53 - DNA, 20 bp, synthetic construct
  misc_feature 1..20: note = Acd_primer_R
  source 1..20: mol_type = other DNA
gtttggacctgggtatggag

SEQ ID NO: 54 - DNA, 21 bp, synthetic construct
  misc_feature 1..21: note = Tbp_primer_F
  source 1..21: mol_type = other DNA
aatataatcccaagcggtttg

SEQ ID NO: 55 - DNA, 20 bp, synthetic construct
  misc_feature 1..20: note = Tbp_primer_R
  source 1..20: mol_type = other DNA
gctgctagtctggattgttc

SEQ ID NO: 56 - DNA, 39 bp, synthetic construct
  misc_feature 1..39: note = telomere_primer
  source 1..39: mol_type = other DNA
cggtttgtttgggtttgggtttgggtttgggtttgggtt

SEQ ID NO: 57 - DNA, 37 bp, synthetic construct
  misc_feature 1..37: note = telomere_primer
  source 1..37: mol_type = other DNA
ggcttgccttacccttacccttacttacccttaccct

SEQ ID NO: 58 - DNA, 23 bp, synthetic construct
  misc_feature 1..23: note = 36B4_primer
  source 1..23: mol_type = other DNA
cagcaagtgggaaggtgtaatcc

SEQ ID NO: 59 - DNA, 25 bp, synthetic construct
  misc_feature 1..25: note = 36B4_primer
  source 1..25: mol_type = other DNA
cccattctatcatcaacgggtacaa